(19) United States
(12) Patent Application Publication
(10) Pub. No.: US 2013/0023914 A1
(43) Pub. Date: Jan. 24, 2013

(54) SYSTEM FOR ACCESSING BODY ORIFICE AND METHOD

(71) Applicants: Stephanie Lily TRUONG, San Francisco, CA (US); Vandana JAIN, Mumbai (IN); Evan LUXON, Stanford, CA (US)

(72) Inventors: Stephanie Lily TRUONG, San Francisco, CA (US); Vandana JAIN, Mumbai (IN); Evan LUXON, Stanford, CA (US)

(73) Assignee: CLEAREAR, INC., San Francisco, CA (US)

(21) Appl. No.: 13/552,604

(22) Filed: Jul. 18, 2012

Related U.S. Application Data
(60) Provisional application No. 61/572,546, filed on Jul. 18, 2011.
(60) Provisional application No. 61/530,337, filed on Sep. 1, 2011.
(60) Provisional application No. 61/615,200, filed on Mar. 23, 2012.

(51) Int. Cl. A61F 11/00

(52) U.S. Cl. 606/162

(57) ABSTRACT

A device for accessing a body orifice is disclosed. The device may include a speculum body having an outer shell and an inner shell nested within the outer shell. A plurality of fluidic channels may be defined in the interstitial space between the inner and outer shells. The outer shell may be configured as an optical waveguide. The inner shell central bore may be configured for viewing and suction. The device may include a speculum body having a tapered shape. The body may be expandable to expand muscles surrounding a body orifice. Programmable controllers, viewing, recording, and communications systems are also described. Methods of using and forming the devices are also described.

### CROSS REFERENCE TO RELATED APPLICATIONS

This application claims the benefit of U.S. Provisional Application No. 61/572,546, filed Jul. 18, 2011, U.S. Provisional Application No. 61/530,337, filed Sep. 1, 2011, and U.S. Provisional Application No. 61/615,200, filed Mar. 23, 2012, each of which is incorporated herein by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This invention relates, in general, to devices for performing diagnosis and/or treatment in a body orifice and methods for their use.

### BACKGROUND

There is a need for performing treatment in body orifices. Conventional medical devices often employ complex and expensive machinery to gain access to the body orifice. As an example, removal of ear wax is a relative low risk procedure yet typically requires several instruments and multiple clinicians.

Ear wax is a normal secretion which becomes a problem in certain circumstances. Ear wax impaction is present in approximately 10 percent of children, 5 percent of normal healthy adults, up to 57 percent of older patients in nursing homes, and 36 percent of patients with mental retardation. The impacted wax is a clinical concern because it can interfere with the clinician's view of the tympanic membrane and cause a conductive hearing loss, hence interfering with formal hearing assessment. If in contact with the tympanic membrane, it can cause discomfort and occasionally vertigo, tinnitus, or chronic cough.

Existing treatment options for ear wax removal vary in terms of efficacy, cost, time, and safety. Ear candling is a popular but ineffective and potentially dangerous homeopathic method of ear wax removal. During ear candling, a 4 to 10-inch hollow candle is placed in the ear canal and one end is lit on fire. The heat allegedly creates suction, which draws earwax out of the ear. Ear candling enthusiasts point to resultant residue as proof of efficacy, but this has been shown to be residue from the candle itself. In addition, studies have shown that no negative pressure is produced, no wax is removed, and that the practice deposits candle wax in the ear.

Similarly, the modern-day cotton-tipped swab (a.k.a. the Q-tip®) is a popular but dangerous method of ear wax removal. Use of cotton-tipped swabs can push ear wax further into the ear, perforate the ear drum, or abrade the ear canal. Kravitz et al. found that 1 in 2000 pediatric office visits and 3 in 1000 ear, nose, and throat (ENT) office visits were solely for cotton-tipped swab injuries. This equates to about 22,000 visits are for injuries caused by cotton-tipped swabs. There are currently no devices for earwax removal with built in safety features to let the user know how far in the ear canal the device is and prevent injuries such as a perforated eardrum.

Cerumenolytic agents dissolve or soften ear wax. Cerumenolytics are often used in combination with other earwax removal tools. They can only break down the outermost surface of the impacted earwax. Thus with the current deliver method for cerumenolytics, their efficacy is limited. There is no current means to continuously deliver cerumenolytics and have them penetrate deeper within the impacted earwax.

Irrigation is the most common type of ear wax removal performed in a primary care physician (PCP) setting and entails expelling fluid from a plastic syringe into the ear canal to manually dislodge and wash out ear wax. One problem with irrigation is that it is currently a blind procedure. Directing a high pressure jet of water without visualization leads to complications such as a perforated eardrum.

ENT physicians use an operating microscope to visualize the ear through an ear speculum. Depending on the type of wax, they then use metal or plastic loops or spoons, alligator forceps, curettes, or right-angled hooks for harder wax or angulated suction for softer wax to remove the ear wax. However, the high level of efficacy and safety is a trade off, as the microscope used by ENT for visualization costs over $9,000, which makes it unaffordable in the PCP setting where the majority of earwax removal procedures are performed. There is currently no inexpensive earwax removal device with outcomes comparable to the ENT.

Although various treatment options exist for earwax removal, each has its shortcomings. There still remands a need a safer, faster, less expensive, and easier to use method of removing earwax.

What is needed are devices that overcomes the above and other disadvantages. What is needed are improved devices that allow access to and operation within body orifices, in various respects, small body orifices.

### SUMMARY OF THE DISCLOSURE

Various aspects of the invention are directed to the devices shown and described.

The devices of the invention have other features and advantages which will be apparent from or are set forth in more detail in the accompanying drawings, which are incorporated in and form a part of this specification, and the following Detailed Description of the Invention, which together serve to explain the principles of the present invention.

In some embodiments, a device for accessing a body orifice is provided. The device includes an outer shell having a proximal end, a distal end, and a central bore; an inner shell coaxial with the outer shell; and one or more fluid channels defined between an outer surface of the inner shell and the inner surface of the outer shell. In some embodiments, the device includes a suction channel. In some embodiments, the outer shell includes a body including an integrated illumination pathway. In some embodiments, the integrated illumination pathway includes a light pipe. In some embodiments, the inner shell comprises a central bore configured as a suction channel. In some embodiments, the inner shell comprises a central bore configured to provide access to the body orifice for a medical instrument. In some embodiments, the medical instrument is a curette. In some embodiments, the device is configured for removal of cerumen from an ear canal. In some embodiments, the inner shell has a longitudinal axis, a proximal end and a distal end, the distal end having one or more fluid guides configured to direct fluid at a predetermined angle away from the longitudinal axis. In some embodiments, the one or more fluid guides is an even number of fluid guides. In some embodiments, each of the one or more fluid guides has a fluid guide surface that is angled at a predetermined angle with respect to the longitudinal axis. In some embodiments, the inner shell comprises a first outer surface and a second outer surface, wherein the second outer surface is offset from the first outer surface such that when the first outer surface of the inner shell is in contact with the inner surface of the outer shell, the second outer surface of the inner shell is offset from the inner surface of the outer shell, thereby forming the one or more fluid channels between the second outer surface of the inner shell and the inner surface of the outer shell. In some embodiments, the one or more fluid channels are in fluid communication with a source of fluid. In some embodiments, the outer shell has a sealing element located on a distal end of the outer shell, the sealing element configured to provide a fluid seal between the device and the body orifice. In some embodiments, the sealing element is reversibly deformable.

In some embodiments, a portable system for removing ear wax from an ear canal of a patient is provided. The system includes a speculum having an inner portion nested within an outer portion, the speculum having one or more fluid channels formed between the inner surface of the outer portion and the outer surface of the inner portion, and an inner bore defined at least in part by the inner surface of the inner portion; a source of fluid in fluid communication with the one or more fluid channels; a vacuum source in communication with the inner bore for removing fluid and ear wax; a pump for transferring fluid from the source of fluid to the one or more fluid channels; an artificial illumination source configured to provide light through the speculum to the ear canal; and a controller in communication with the pump for delivering fluid to the ear canal according to a predetermined treatment routine. In some embodiments, the system further includes a tool or curette configured to pass through the inner bore of the speculum. In some embodiments, the tool or curette comprises an elongate body having a distal end and a wire loop extending away from the elongate body. In some embodiments, the tool comprises a wheel actuator that is configured to extend and retract a portion of the tool by rotation of the wheel actuator. In some embodiments, the wheel actuator comprises a wheel with a circumferential groove and an elongate and elastic member wrapped around the circumferential groove. In some embodiments, the elongate body is hollow and in communication with a vacuum source, the elongate body having a suction port located at the distal end of the elongate body. In some embodiments, the wire loop is located proximally the suction port. In some embodiments, the artificial illumination source is one or more light emitting diodes. In some embodiments, the system further includes a handle adapter, wherein the handle adapter is reversibly connected to the speculum and provides a gripping surface. In some embodiments, the handle adapter has a viewing window aligned with the inner bore of the speculum. In some embodiments, the controller and artificial illumination source are located on the handle adapter. In some embodiments, the speculum comprises a suction port located on the bottom portion of the speculum, the suction port configured to provide communication between the inner bore and the vacuum source. In some embodiments, the system further includes a compact housing that holds the source of fluid, the vacuum source, and the pump. In some embodiments, the compact housing is between about 6 to 18 inches in length, 6 to 18 inches in width, and 4 to 12 inches in height. In some embodiments, the system further includes a heating element configured to heat the fluid to a predetermined temperature.

In some embodiments, a method of removing ear wax from an ear canal of a patient is provided. The method includes inserting a speculum having a longitudinal axis into the ear canal; illuminating the ear wax within the ear canal with an artificial light source; viewing the illuminated ear wax through the speculum; irrigating the interior of the ear canal with one or more jets of irrigation fluid, wherein the one or more jets of irrigation fluid are ejected towards the ear canal walls from the speculum at a predetermined angle away from the longitudinal axis; dislodging at least a portion of the ear wax with the irrigation fluid while viewing the illuminated ear wax; and suctioning the irrigation fluid and the dislodged portion of the ear wax from the ear canal. In some embodiments, the method further includes inserting a tool or curette through the speculum to dislodge at least a portion of the ear wax. In some embodiments, the method further includes suctioning at least a portion of the ear wax with the tool or curette. In some embodiments, the predetermined angle is between about 5 and 45 degrees. In some embodiments, the ear canal is irrigated with about 300 to 700 mL/min of irrigation fluid. In some embodiments, the irrigation fluid is delivered at less than 5 psig. In some embodiments, the irrigation fluid is heated to about 37 degrees Celsius before being used to irrigate the ear canal. In some embodiments, the method further includes actively drying the ear canal after irrigation. In some embodiments, the step of drying includes introducing a jet of air into the ear canal. In some embodiments, the method further includes creating a negative pressure within the interior of the speculum.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a perspective view of an exemplary device for removal of ear wax in accordance with various aspects of the invention.

FIG. 2 is an exploded view of the device of FIG. 1.

FIGS. 3 and 4 are side views of the outer shell of the speculum of FIG. 1.

FIG. 5 is a perspective view of the outer shell of FIG. 1.

FIG. 6 is an enlarged end view of the outer shell of FIG. 1.

FIG. 7 is a perspective view of the inner shell of FIG. 1.

FIGS. 8 and 9 are side views of the inner shell of FIG. 1.

FIG. 10 is an enlarged end view of the outer shell of FIG. 1.

FIG. 11 is a perspective view of the cap of FIG. 1.

FIGS. 12 and 14 are side views of the cap of FIG. 1.

FIG. 13 is a top view of the cap of FIG. 1.

FIG. 15 is a perspective view of an exemplary device similar to the device of FIG. 1, illustrating a wire frame speculum body.

FIG. 16 is a side view of an exemplary collection plate for use with the device of FIG. 1, illustrating the collection plate on a patient's ear.

FIG. 17 is a perspective view of the pump system for use with the device of FIG. 1.

FIG. 18 is a schematic of the pump system of FIG. 17.

FIG. 19 is a back view of the pump system of FIG. 17.

FIGS. 20-26 are various views of an exemplary device similar to that of FIG. 1 configured for accessing the uterus through the vaginal tract.

FIGS. 27-28 are perspective views of an exemplary tool for use with the device of FIG. 1. FIG. 27 illustrates a retracted position. FIG. 28 illustrates an extended operating position.

FIGS. 29A and 29B are various views of an exemplary embodiment of an ear wax removal system.

FIG. 30 is a perspective view of a portion of the ear wax removal system of FIG. 29 separated in individual components.

FIG. 31 is a perspective view of the portion of the ear wax removal system of FIG. 30 with the components assembled together.

FIG. 32 is a side view of the ear wax removal system inserted into the ear canal of a patient.

FIG. 33 is a perspective view of an embodiment of a curette that can be used in the ear wax removal system illustrated in FIG. 29.

FIGS. 34A-34C are various views of an embodiment of a speculum and handle adapter in use with a curette.

FIG. 35A and 35B are various views of embodiments of a speculum.

FIGS. 36A-36D are various views of an embodiment of the speculum.

FIG. 37 is a perspective view of an embodiment of the speculum attached to a handle adapter.

FIGS. 38A and 38B are various views of an embodiment of a light pipe that focuses light produced by the handle adaptor through the speculum.

FIG. 39 is a front view of another embodiment of an ear wax removal device.

FIG. 40 is a perspective view of the device of FIG. 39 in the retracted position.

FIG. 41 is a right view of the device of FIG. 39 in the retracted position.

FIG. 42 is a perspective view of the device of FIG. 39 in the actuated position.

FIG. 43 is a front view of yet another embodiment of an ear wax removal device.

FIG. 44 is a perspective view of the device of FIG. 43.

FIG. 45 is a perspective view of the inner piece of the device of the device of FIG. 43 with a flared tip.

FIG. 46 is a perspective view of the outer piece of the device of FIG. 43 with a flared tip.

FIG. 47 is a sketch of the device of the FIG. 43.

FIG. 48 illustrates the delivery of irrigation fluid from the device of FIG. 43.

FIGS. 49A-49C illustrate other embodiments of a suction curette.

FIGS. 50A and 50B illustrate other embodiments of a suction tube.

FIG. 51 illustrates an embodiment of a curette with a cutting tool.

FIGS. 52A and 52B illustrate embodiments of a fluid delivery tool.

### DETAILED DESCRIPTION

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims.

For convenience in explanation and accurate definition in the appended claims, the terms “up” or “upper”, “down” or “lower”, “inside” and “outside” are used to describe features of the present invention with reference to the positions of such features as displayed in the figures.

In many respects the modifications of the various figures resemble those of preceding modifications and the same reference numerals followed by subscripts “a”, “b”, “c”, and “d” designate corresponding parts.

Depending on severity of earwax impaction, earwax removal treatment can vary from requiring only 5 minutes to as long as 45 minutes, depending on the skill level of the person performing the procedure. The device has been specifically designed to be incorporated into settings in which earwax removal procedures are performed, but limited time and qualified personnel are available to perform them. As an example, the earwax removal device may be incorporated within the safety net system, in which the hospitals and clinics in this system are operating under a tight budget and have limited capacity. Safety net facilities share “a common mission to serve everybody who walks through their doors, regardless of their ability to pay.” Currently, many safety net clinics are not able to meet this goal with regard to earwax removal since the procedure takes 45 minutes.

ENTs are highly trained and are capable of removing earwax the most safely and quickly. Earwax removal is the most common ENT procedure. Clearly the opportunity cost of earwax removal for ENTs is very high. Trained sub-specialists in Otolaryngology want to greatly enhance clinical outcomes of patients with much higher mortality or morbidity risks, but are stuck removing earwax. In the safety net setting, the goal is to do the most with the least amount of money and resources so there is a need to treat earwax removal procedures in the PCP setting, rather than sending patients to the ENT specialists. Primary care physicians (PCPs) at these facilities are highly limited on time that they can spend with each patient.

In addition to the time required to actually remove earwax, clinicians are advised to spend extensive time assessing the patient for pathophysiologies that might make irrigation unsafe, such as one or more of the following: nonintact tympanic membrane, ear canal stenosis, exostoses, diabetes mellitus, immunocompromised state, or anticoagulant therapy. Despite the high minded guidelines however, 80% of PCPs have their nurse or medical assistant remove earwax, sometimes without even visually confirming earwax impaction themselves. Partly because PCP offices, especially in the safety net setting, do not have time to follow the rigorous guidelines for earwax removal, major complications (of the level that may result in a lawsuit) and in some cases result in permanent hearing loss occur at a rate of approximately 1 in 1000.

In the safety net setting, most earwax removal procedures must be performed by lower-skilled personnel due to constraints. PCP/Nurse/Assistants, and ENTs have a strong positive interest in a safer, faster, easier method of removing earwax in the PCP setting. Additionally, removing earwax in all patients before they see an audiologist has the potential to improve specificity of the audiology exam and improves coordination between the PCP and audiologist. With the device of the following embodiment, more earwax removal procedures can be performed with fewer complications and less time, ultimately increasing the efficiency of the safety net system and the quality of care it provides. In some embodiments, the systems, devices and methods described herein can be used and performed by one or more of a PCP, Nurse, Assistant, ENT, and Audiologist.

The device of any of the following embodiments may also be incorporated at earwax clinics and the private (non-safety net) system, where many earwax removals are frequently preformed. Patient discomfort during the earwax removal procedure can be extensive, especially if the earwax is particularly hard to remove. Patients may complain of pain, infection, dizziness, tinnitus or hearing loss. The device of the following embodiment may decrease the discomfort experienced during an earwax removal procedure by decreasing the overall time of the procedure and/or decreasing the amount of discomfort caused by the procedure performed with the device. Furthermore, the device of the following embodiment also seeks to provide a more user-friendly experience for the person performing the procedure.

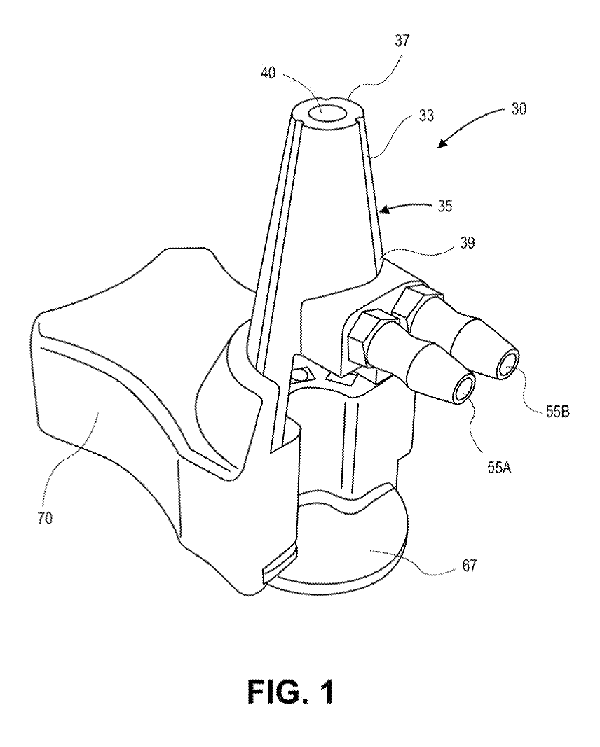

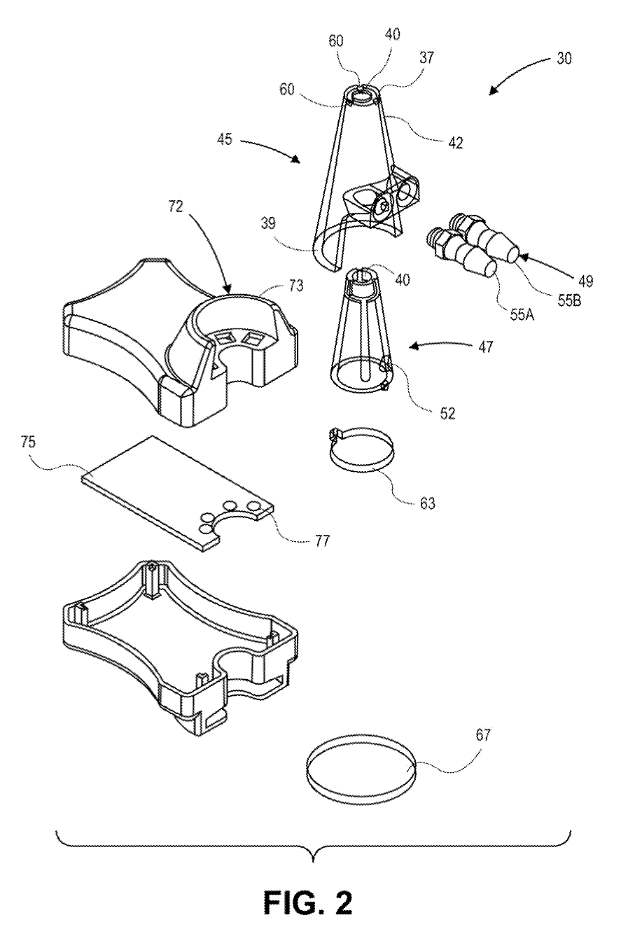

Turning now to the drawings, wherein like components are designated by like reference numerals, attention is directed to the various figures. FIGS. 1 and 2 show a device, generally designated 30, for accessing a body orifice. In various respects, body orifice and body cavity are used somewhat interchangeably. In various embodiments, the device is part of a system 28 for accessing a body orifice. Various aspects of the device are similar to the ones described in U.S. patent application Ser. No. 13/150,915 to Stephanie Truong, the entire contents of which are incorporated herein for all purposes by this reference.

The exemplary device is configured for providing a user access to body orifice for diagnosis, therapy, or both. In various respects, the device is configured for access to a small body orifice. Typically the cavity presents an external orifice. In various respects, the body orifice and/or cavity are internal. For example, the device may be configured for percutaneous insertion or open surgery.

In various embodiments, the body orifice is a member selected from an ear canal, the urinary tract, the vaginal tract, and the anus and gastrointestinal/alimentary tract.

In various embodiments, the body orifice is an ear canal. In various embodiments, the orifice is a human ear canal. The human ear canal leads from an orifice in the fibrocartilaginous external part of the ear and pinna to an ear drum. The outer ear is sometimes referred to as the outer ear. Inward of the ear drum are the features for listening such as the inner ear consisting of the cochlea and vestibular system, the auditory ossicles for transmitting sound to the cochlea, the Eustachian tube (auditory tube that links the pharynx to the middle ear), and the auditory nerve. A typical human ear canal is about 35 mm in length and about 5 mm to about 10 mm in diameter. The ear canal may be round or oval shaped.

In various embodiments, the device is configured for treating a disorder selected from atresia, otitis externa (swimmer's ear), inflammation, bacterial and fungal infection, contact dermatitis of the ear canal, ear myiasis (infestation of maggots), undesirable bone exposure in the canal, abnormal tissue disorders, granuloma, stenosis or blockage of the canal, a foreign body in ear (e.g. objects accidentally inserted into a child's ear), cholesteatoma, and ear mites.

FIG. 2 is an exploded view of device 30. The exemplary device includes a working part and a body 33. The working part comprises a speculum 35.

In various respects, speculum 35 is shaped and dimensioned similar to a conventional otoscope or auriscope working end. In various respects, the speculum is similar to the ones disclosed in U.S. patent application Ser. No. 13/150,915 to Stephanie Truong, the entire contents of which are incorporated herein for all purposes by this reference.

The exemplary speculum 35 is shaped for insertion into an ear canal. The speculum has a frustoconical shape. The speculum has a distal end 37, a proximal end 39, and a bore 40.

Distal end 37 of the exemplary speculum is dimensioned to correspond to the ear canal diameter. In various embodiments, the distal end has a diameter of about 6 mm, about 7 mm, about 8 mm, about 9 mm, about 10 mm, or about 11 mm. The exemplary distal end of the speculum has an outer diameter of about 8 mm and a bore diameter of about 4 mm. It may be desirable to maximize the diameter of the distal end while still allowing insertion into the ear canal. In various embodiments, a system is provided with a plurality of speculums each having a different shape and/or size.

The speculum flares in a direction moving from the distal end to the proximal end. The speculum body may increase linearly from the distal end to the proximal end to form a conical shape.

Exemplary speculum 35 comprises an outer shell 45 and an inner shell 47. The outer shell defines the outer surface of speculum 35. The outer shell is configured to receive the inner shell. In various embodiments, the outer shell is configured receive the inner shell in a nested configuration.

A tip portion 42 of outer shell 45 is configured for opening up and/or straightening the ear canal to improve access. The tip portion widens moving from a distal most end towards proximal end 39. Thus, the tip portion can be lodged in the ear canal and maintains the canal opening. The exemplary tip portion is linearly aligned to straighten the ear canal. “Linearly aligned” refers to the relatively straight central longitudinal axis of the respective element.

The exemplary speculum 35 is configured for removal of cerumen (ear wax). The exemplary speculum is configured for irrigation, suction, or both. The speculum also provides access to the body orifice for instruments, viewing, and more. In various embodiments, device 30 is configured to allow irrigation, suction, and viewing while inserted in the body orifice.

The tip portion defines an enclosed bore 40. The bore 40 is open at an opposite, proximal end of the speculum. The tip portion is dimensioned to extend into the ear canal opening.

Exemplary bore 40 extends from the distal end to the proximal end; however, one will appreciate from the description herein that the bore may extend less than the length of the speculum. The bore may have a constant diameter or a variable diameter along its length.

Speculum 35 may be configured for viewing, illumination, and/or magnification. In various embodiments, the outer shell is configured as an optical waveguide. The outer shell may include a fiber optic line or other structure defining an optical pathway. In various embodiments, the outer shell wall is configured to define the light pathway. For example, the outer shell may be configured for total internal reflection (TIRF). The exemplary outer shell is configured so light propagates from the proximal end to the distal end where it illuminates the ear canal. The exemplary outer shell has a generally planar wall shape to promote TIRF. In this manner, the speculum provides illumination without taking up extra space in ear canal.

Similarly, the speculum may be configured for viewing and/or magnification through bore 40. In practice, a user places his/her eye to the proximal end of the speculum to view the ear canal through the bore.

Exemplary device 30 has a nested design configured to define a plurality of channels. Inner shell 47 is nested within outer shell 45 in close fitting relationship. The exemplary channels are formed at least in part in the space between the inner shell and outer shell. In various embodiments, the device includes a plurality of fluid channels defined between the inner and outer shells. The channels may be configured for viewing, suction, irrigation, or a combination of the same. The exemplary device includes one or more fluid channels for irrigation. In various embodiments, suction is provided through the relatively large diameter bore 40. The exemplary irrigation channel and suction channels generally have a circular cross-section, but one will appreciate that other shapes may be appropriate. For example, it may be desirable to modify the shapes at a portion along the flowpath to reduce dead space thereby increasing utilization of the body cavity space.

Outer shell 45 includes a fitting for receiving a manifold 49. The exemplary outer shell includes two ports 55a and 55b. Exemplary inner shell 47 includes a groove 50 along an outer surface and a through hole 52. When the inner shell is inserted into the outer shell, an end of groove 50 aligns with port 55a to define an irrigation channel. An opposite end of the irrigation channel outlets at a distal end of the speculum. In various embodiments, fluid is directed from the groove channel through ejector holes 60 in the outer shell. The speculum may be configured to direct the fluid to the ear canal in jets at an angle to the ear canal axis. In an exemplary embodiment, holes 60 are positioned at an angle to the ear canal axis. In various embodiments, the angle of the holes is about 30 degrees, about 45 degrees, about 60 degrees, or a combination of the same with respect to the canal axis. The exemplary holes 60 have a diameter of about 1.25 mm. One will appreciate that the irrigation fluid outlet may be modified to control fluid delivery and the jet function. Suitable irrigation fluid includes, but is not limited to, saline solution, water, a lipid solution, hydrogen peroxide solution, and more. The irrigation fluid may be warmed or cooled before delivering to the ear canal.

The irrigation function serves to break up impacted cerumen in the ear canal. In various embodiments, the device is configured to deliver irrigation fluid to the ear canal in a non-continuous flow. In various embodiments, the device is configured to deliver a pulsatile flow. The fluid may be delivered in alternating high pressure and low pressure. The fluid may be delivered alternating bursts of jets and no fluid flow. The pressure of the fluid delivered to the ear canal over time may be sinusoidal or saw shaped. As high pressure risks injuring the ear canal (e.g. perforations), it may be desirable to limit the maximum fluid pressure in the ear canal. In various embodiments, the maximum fluid pressure delivered to the ear canal is 5 psi. In various embodiments, the maximum fluid pressure delivered to the ear canal is 3 psi.

Through hole 52 aligns with port 55b. Thus, port 55a is in fluid communication with bore 40. The suction channel extends between port 55a and a distal end of bore 40.

In an exemplary embodiment, ports 55a and 55b are positioned between proximal end 39 and distal end 37. Thus, the removed material exits closer to the distal tip. This reduces interference of the irrigation and/or suction channels with bore 40. In turn, this improves viewing through the bore and access to the ear canal with an instrument.

In an illustrated embodiment, ports 55a and 55b have the same diameter. The exemplary ports are each 2 mm in diameter. The ports may have different diameters. The suction channel can become obstructed by bits of removed ear wax. By contrast, the irrigation channel flows low viscosity fluid. Accordingly, it may be desirable to have a larger flow pathway for the suction channel than the irrigation channel.

The flow dynamics through the fluid channels are defined in part by the minimum diameter of the channel, wall surface, and bends in the channel. In various embodiments, the speculum is formed of materials to define a relatively smooth wall surface for the channels. In various embodiments, the fluid channels are prepared for smoother flow such as by adding a surface treatment (e.g. coating with a low friction material), heat treatment, and the like.

Outer shell 45 includes an aperture 58 for receiving a corresponding alignment pin 60 on inner shell 47. The pin and aperture provide rotational and translational indexing between the inner and outer shells. In various embodiments, the device is provided to a user in an assembled state. The aperture allows the user to eject the inner shell from the outer shell.

Suction is provided through port 55b and into bore 40. To keep a vacuum, a cap 63 is provided over a back end of tip portion 42. The cap seals off the portion of the bore extending through the tip. The cap may be removed when suction is not desired. The cap may be removed to open the bore and ear canal to ambient pressure. A portion of the cap may be removed to allow access into the tip portion and ear canal. An example of a cap with a cut-away section is disclosed in U.S. patent application Ser. No. 13/150,915 to Stephanie Truong, the entire contents of which are incorporated herein for all purposes by this reference.

In various embodiments, cap 63 is a lens for viewing and/or magnification. The exemplary device includes a back lens 67. The cap lens allows for improved magnification. For example, the two lenses can be configured in a telescope configuration. With the dual lens configuration, magnification can reach and even exceed 50× with simple materials. In various embodiments, the lenses are removable such that one can easily select a desired magnification. The position of the back lens and cap may be adjustable to provide further magnification adjustment.

One will appreciate that this configuration allows for improved magnification and viewing in a relatively simple, cheap, and compact design. Conventional otoscopes require attaching a speculum over a tip. Thus, the lens system in the otoscope is positioned remotely from the ear canal. Conventional otoscopes typically have an expensive lens system. Moreover, conventional otoscopes are configured for viewing only (i.e. diagnostic functionality). The user has to remove the otoscope and insert a different instrument for suction, irrigation, and other functions.

Attention is now directed to generally to FIGS. 2-14. As described above, outer shell 45 has a sectioned body whereby a portion of the interior is open. The outer shell body has a tip portion 42 that is closed. A portion of the circumference of the body is cut out at an opposite end. The inner shell 47 is dimensioned to be received in tip portion 42. The length of the outer shell corresponds to the length of the tip. Thus, when the inner shell and outer shell are assembled, the proximal end of the bore extending through the outer shell is open.

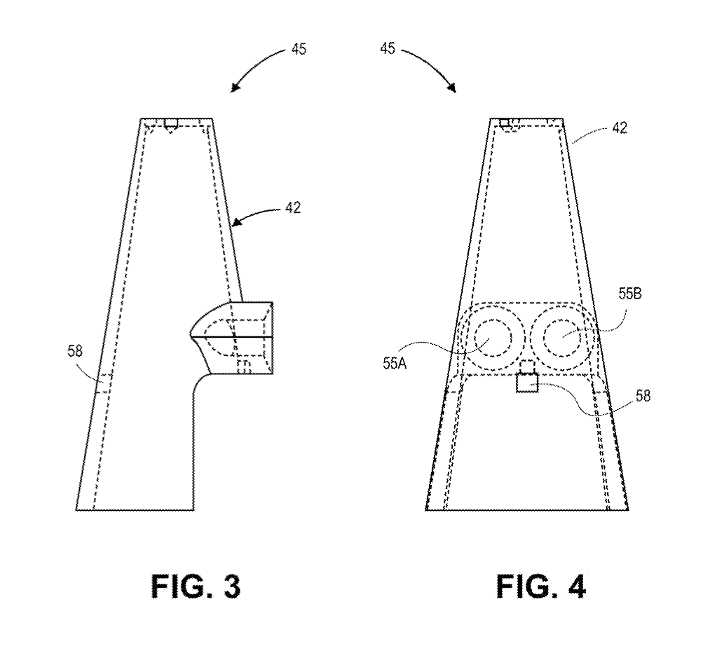

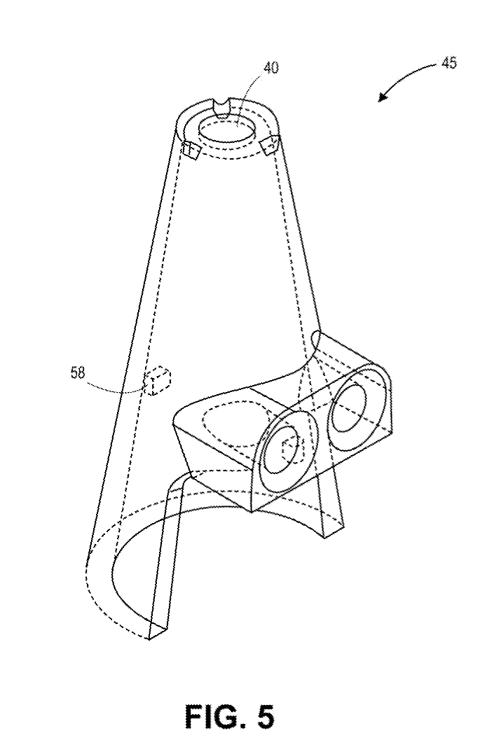

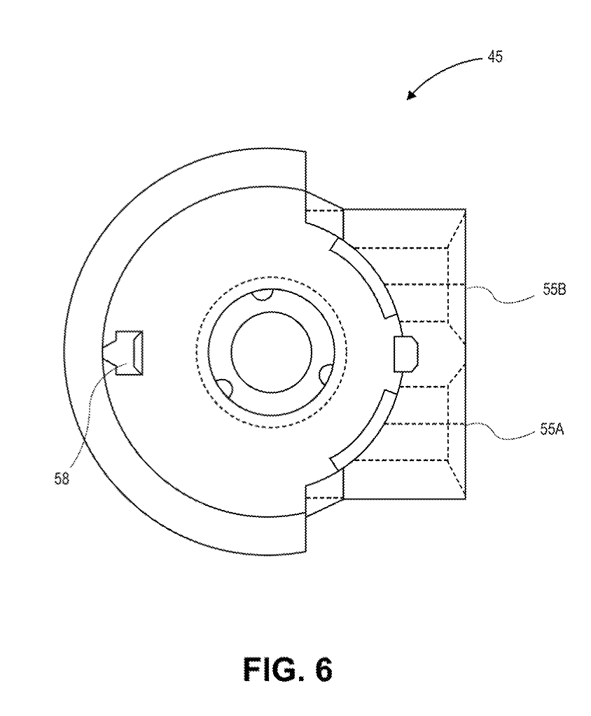

FIGS. 3-4 are side views of exemplary outer shell 45. FIG. 5 is a perspective view of the outer shell. FIG. 6 is an end view of the outer shell as viewed looking from the proximal end to the distal end. In various embodiments, the outer shell has a length of about 43 mm. In various embodiments, the outer shell has a maximum radius of about 11 mm, and in various respects, 11.102 mm. In various embodiments, the maximum radius is at the proximal most end. In various embodiments, tip portion 42 extends from the distal most end and has a length of about 27 mm or about 28 mm.

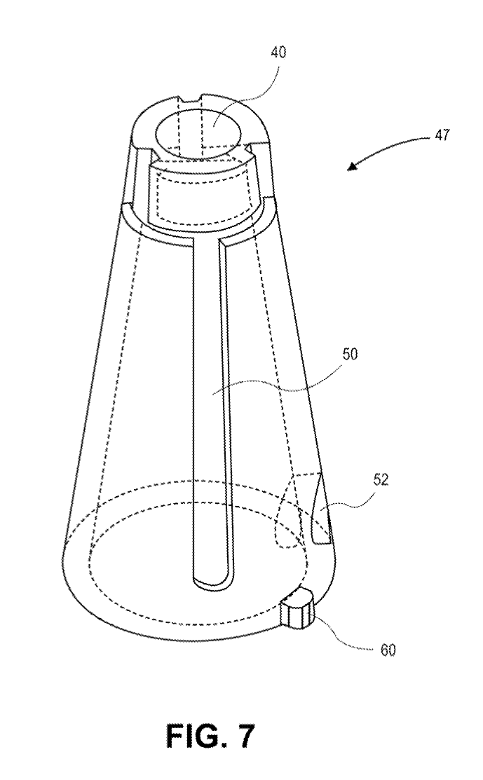

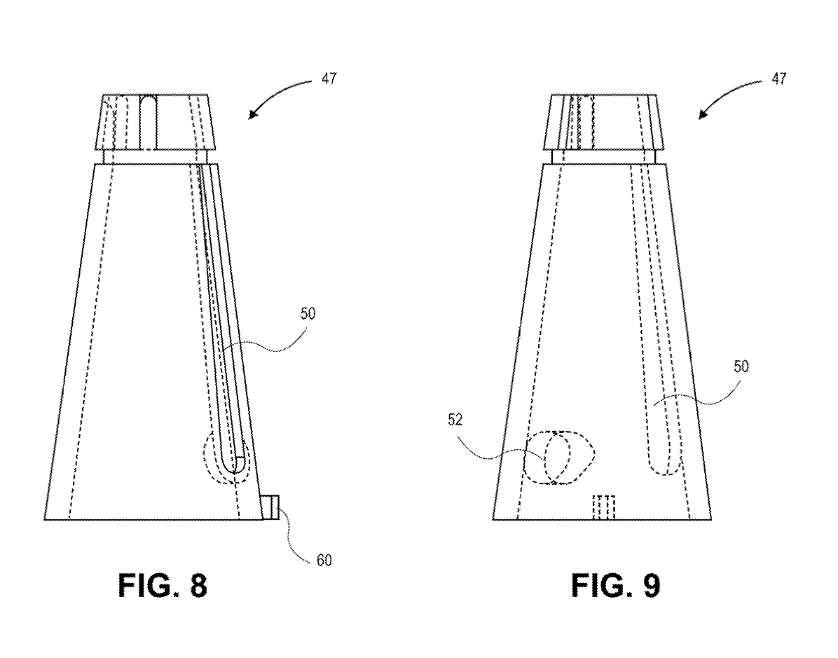

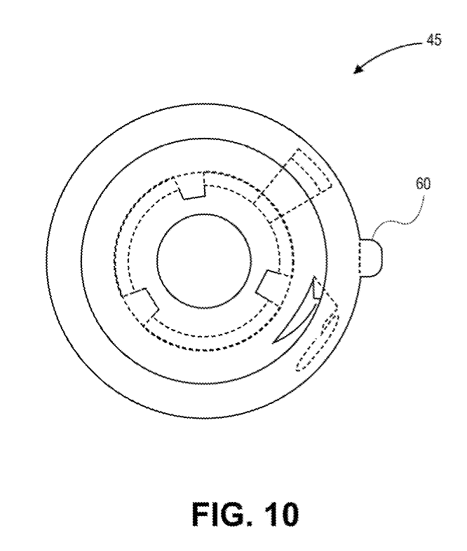

FIG. 7 is a perspective view of the inner shell, and FIGS. 8 and 9 are side views. FIG. 10 is an end view of the inner shell as viewed looking from the proximal end to the distal end. In various embodiments, the inner shell has a length of about 27 mm, and in various respects, 27.437 mm. In various embodiments, the inner shell has a maximum radius of about 7 mm, and in various respects, 7.101 mm. In various embodiments, the maximum radius is at the proximal most end.

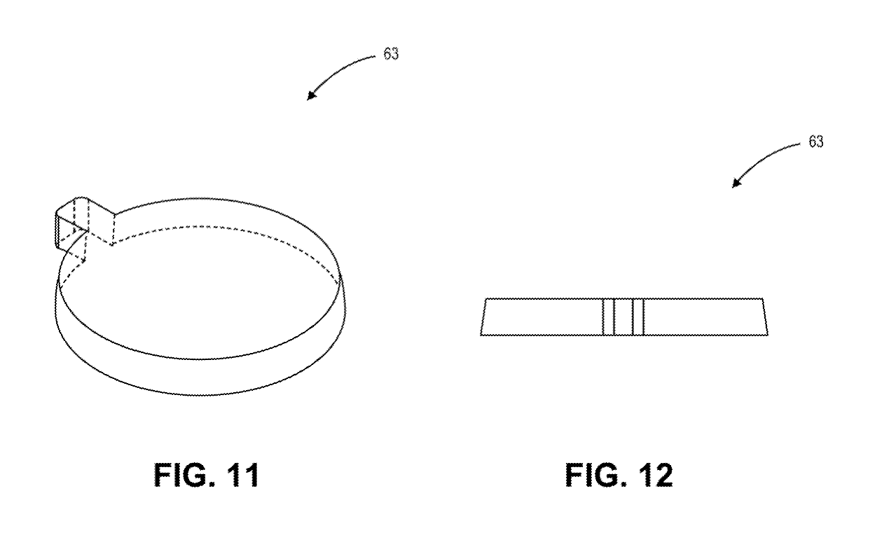

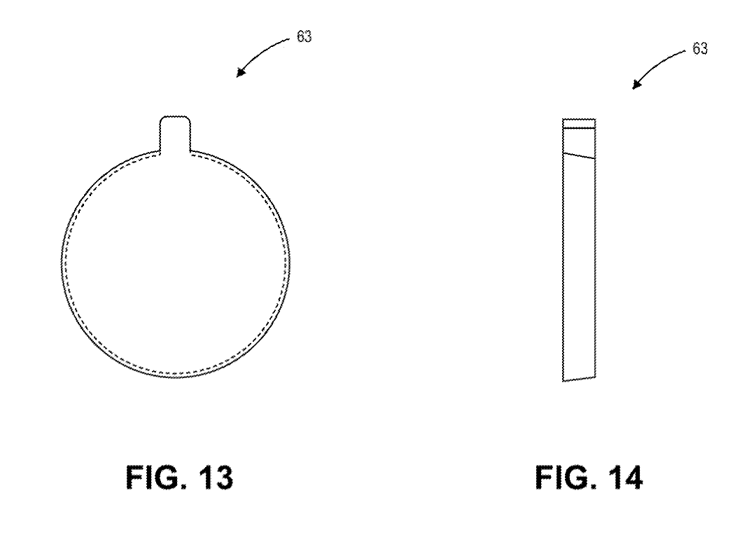

FIG. 11 is a perspective view of exemplary cap 63. FIGS. 12 and 14 are side views of the cap. FIG. 13 is a top view of the cap. In various embodiments, the cap has a thickness of 2 mm. In various embodiments, the diameter of the cap through alignment pin 60 is about 7 mm, and in various respects, 7.368 mm.

As can be appreciated from FIGS. 1 and 3, the non-circumferential proximal end of the outer shell allows for a relatively large access to bore 40. In practice, a user can more easily navigate a tool or instrument into the open area to access the ear canal through the bore.

Device 30 includes a handle 70. The handle comprises a body for supporting the working elements, for example, speculum 35. The exemplary handle body includes a mounting portion 72 having a lip 73 on a front face. The mounting portion mounts the speculum, and in an exemplary embodiment, the distal end of the outer shell. The exemplary lip extends more than 180 degrees to provide a snap fit for the speculum.

A back face of the body includes a lens receiving slot for holding lens 67. The slot is dimensioned to receive and fix the lens in an axially aligned position with the speculum. The lens receiving slot extends more than 180 degrees to provide a snap fit for the lens. Other attachment mechanisms may be used for the speculum and lens.

The exemplary handle body is a two-piece case. A printed circuit board (PCB) 75 is enclosed within the two pieces. The PCB is programmed to control device 30. A field programmable gate array (FPGA), microprocessor, microcontroller, and/or memory or other device may be used instead of a PCB.

PCB 75 has a cut-out section corresponding to the shape of the speculum so it does not interfere with the speculum function. A plurality of light sources 77 are mounted on the PCB. In an exemplary embodiment, the light sources are light emitting diodes (LED). The LEDs are mounted around the cut-out section so the emitted light is directed into the illumination pathway (e.g. wall) of outer shell 45. A power source (e.g. battery), switch, and USB are provided on board the PCB.

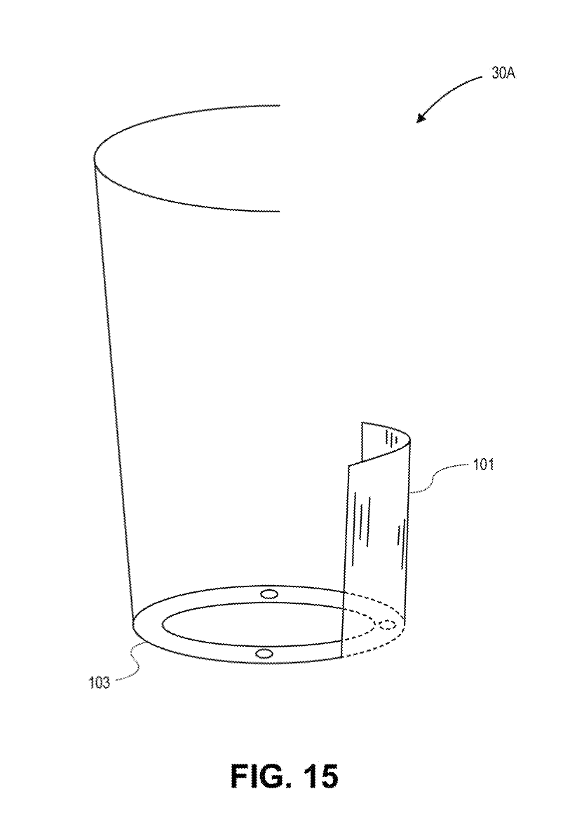

FIG. 15 illustrates a device 30a similar to device 30. Unlike device 30 with a solid outer shell, device 30a includes a wireframe body. One portion of the body is formed of a wire. Suitable materials include, but are not limited to, medium gauge steel and nitinol. The body may include a fiber optic wire or other suitable light-guiding structures and materials. Another portion of the body includes a wall 101. The wall is dimensioned and configured similar to tip portion 42 and defines a suction and/or viewing port. A distal end of the body is formed of an annular base 103. The base may be a rigid ring or other suitable structure. The exemplary base includes three holes for directing irrigation fluid into the ear canal.

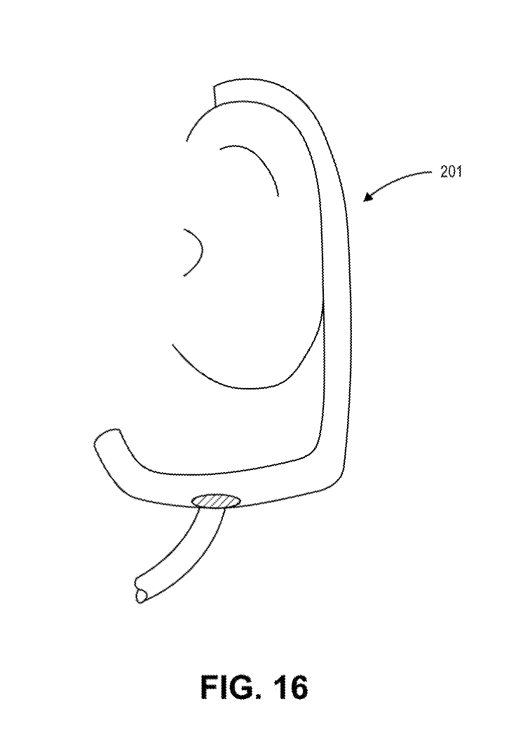

FIG. 16 illustrates a collection tray, generally designated 201, for use with devices 30 or 30a. The collection tray is shaped to attach to the ear cartilage. By contrast, the patient typically is required to hold the collection tray using conventional wax removal techniques. The illustrated collection tray includes a bottom portion extending below the collection tray. The bottom portion is configured to collect wax and other materials removed from the ear canal. The collection tray includes an aperture and tubing for transferring collected material back into the suction pathway. This allows the fluid to be recycled in the system.

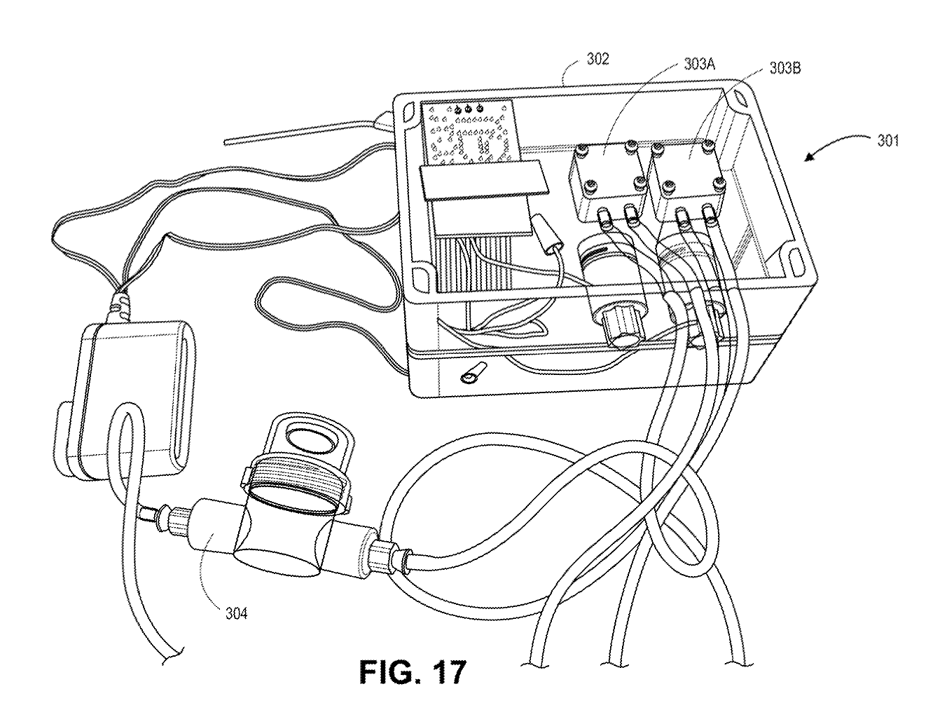

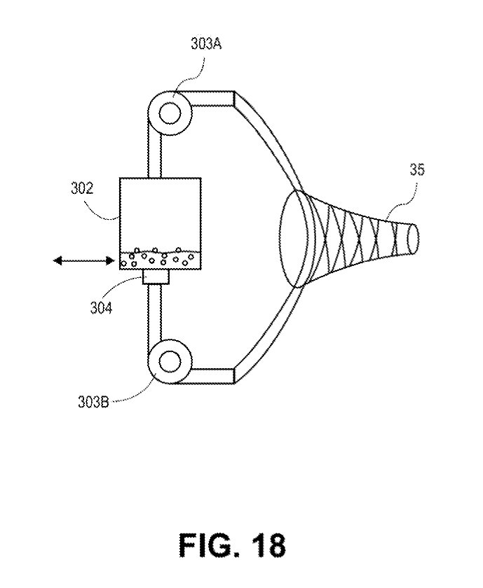

FIGS. 17-19 illustrate a pump system 301 for use with devices 30 or 30a. The exemplary pump system is a closed loop system so the fluid is recycled. This reduces the need for the clinician to refill the system. Conventional systems require refilling the fluid 5, 10, and even 15 times for each patient. The pump system of the invention thus significantly reduces procedure time.

The exemplary pump system includes two pumps within a housing 302. A first pump 303a pushes irrigation fluid to speculum 35. A second pump 303b provides suction or aspiration. The use of two or more pumps reduces pressure loss. By providing separate pumps for the irrigation and suction functions, the system also allows each function to be selectively turned on and off. In one embodiment, the user does not need suction and therefore turns off the suction pump. For example, the user may be using the speculum with a curette tool and not desire irrigation and/or suction.

The exemplary pumps are peristaltic pumps. The pump system includes quick-connects to allow easy removal of tubing from pump housing 302. In one embodiment, the pumps are diaphragm pumps.

As described above, the pump system is configured for pulsatile jet flow. The pumps may be pulsed on and off by PCB 75. In some embodiments, the pumps can directed to provide pulsatile irrigation at about 0.5 to 100 pulses/second, or about 0.5 to 50 pulses/second, or about 0.5 to 10 pulses/second, or less than about 10 pulses/second, or less than about 5 pulses/second, or greater than about 0.5 pulses/second, or greater than about 5 pulses/second. The pumps may be configured specifically for pulsatile flow. The pulsatile flow has been shown to improve fluid pressure without a commensurate increased risk of damage to the ear drum. Pulsatile flow techniques have also been shown to safely increase in the overall fluid flow (mL/min) to the ear canal. In various embodiments, the irrigation pressure threshold is 5 psig. In various embodiments, the pumps have a maximum outlet pressure of about 0.1 MPa (about 14 psig).

In various embodiments, PCB 75 is pre-programmed with a routine function. The PCB may include programming for performing various treatment protocols. For example, the system can be pre-programmed to start at high pressure then slowly decrease to a stop after a set time. After the set time, if wax not removed, user knows that the current treatment will not work. In this case, the user may be directed to return at a later time or the clinician can switch to a different technique, for example, use a curette tool to create an opening in the impacted ear wax. In one example, the treatment protocol involves turning on for a set period of time such as three minutes. In various embodiments, the PCB is programmed to maintain irrigation pressure and/or suction pressure below predetermined thresholds.

In various embodiments, the pump system is configured to have a continuous flow rate. In various embodiments, the pump system is configured to have a non-continuous flow rate. In various embodiments, the pump system is configured to have a flow rate of about 300 mL/min, about 400 mL/min, about 500 mL/min, about 600 mL/min, about 700 mL/min, or greater than 700 mL/min. In various embodiments, the pump system is configured to have a non-continuous flow rate of about 600 mL/min. It has been found that a relatively high, non-continuous flow increases efficacy without affecting safety.

The pump system 301 includes a filter for filtering particles from the fluid taken out of the ear canal. This allows the system to recycle the fluid flow. The exemplary system includes an in-line filter 304. The filter may be a wire mesh or other known filtering device.

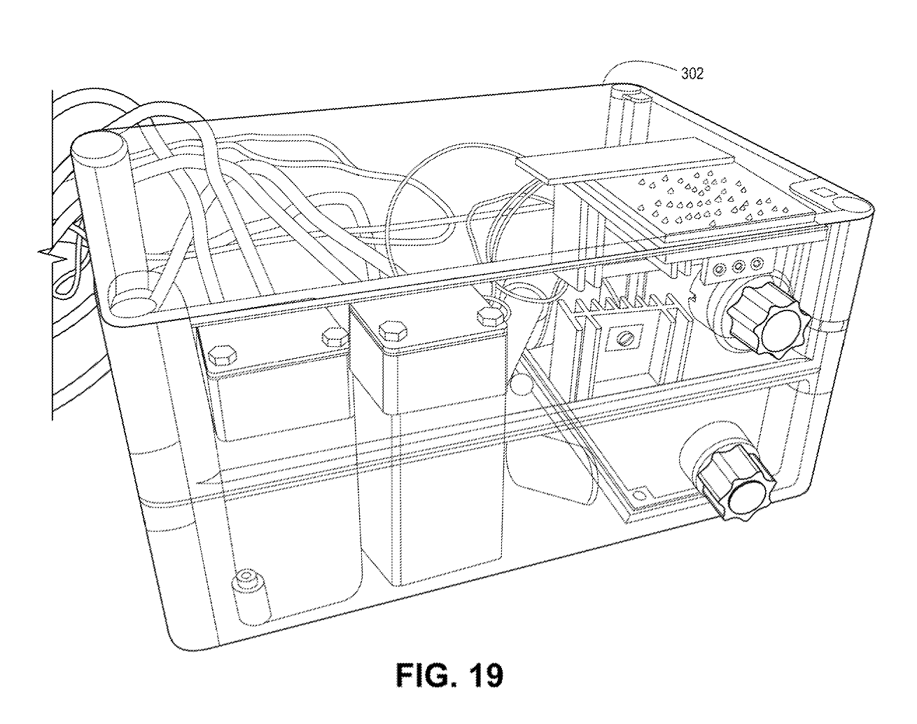

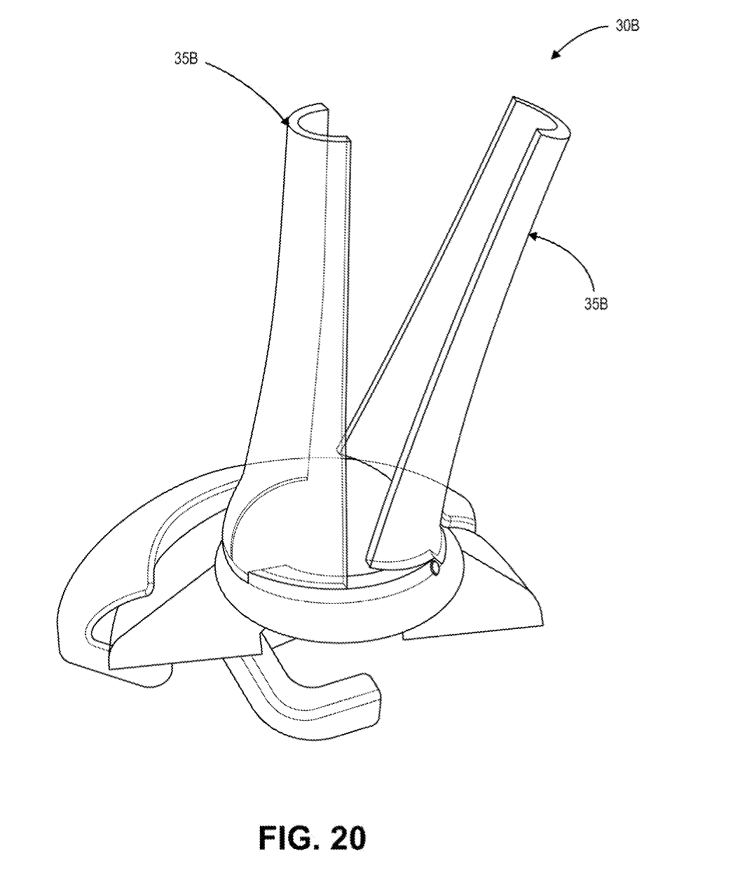

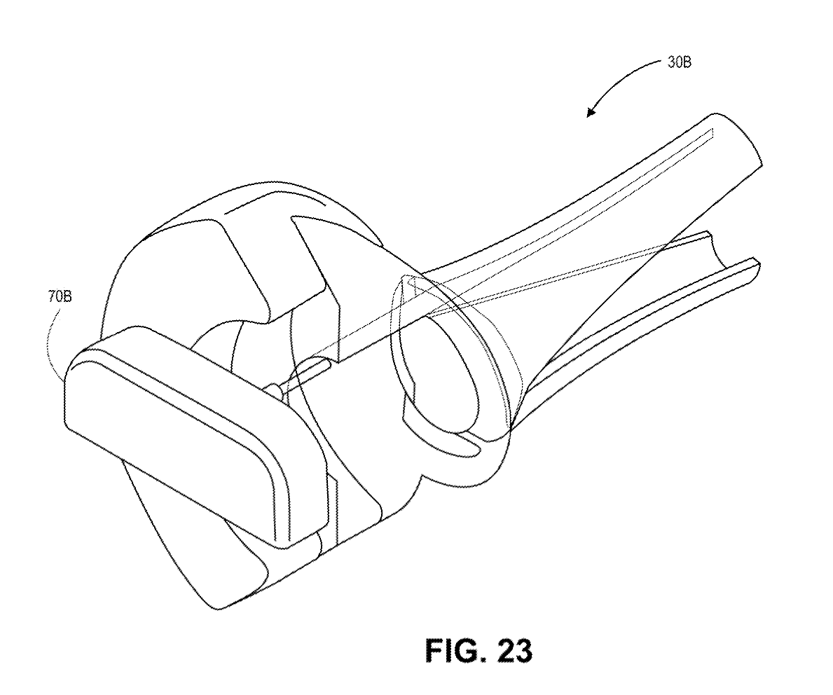

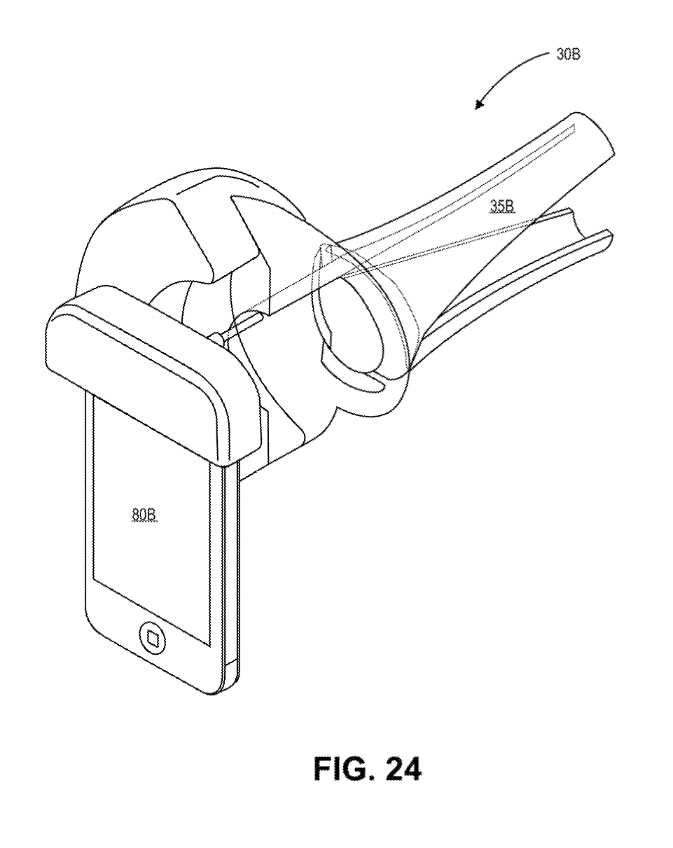

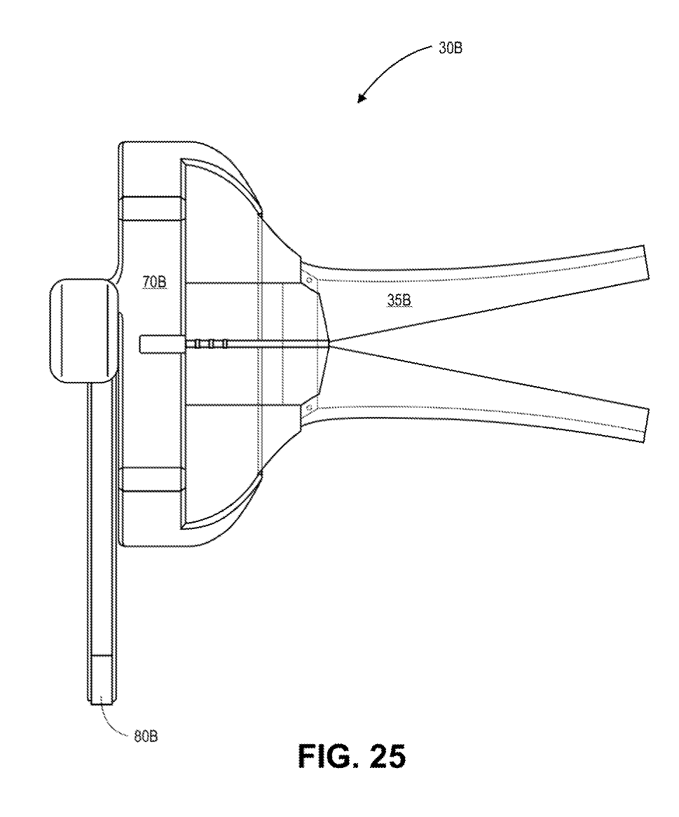

FIGS. 20-26 illustrate a device 30b similar to devices 30 and 30a. Device 30b is configured for accessing a vaginal canal, for example, to examine or treat the cervix. The device includes a speculum 35b for insertion into the vagina. By contrast to speculum 35, speculum 35b is larger to gain access to the larger vaginal canal.

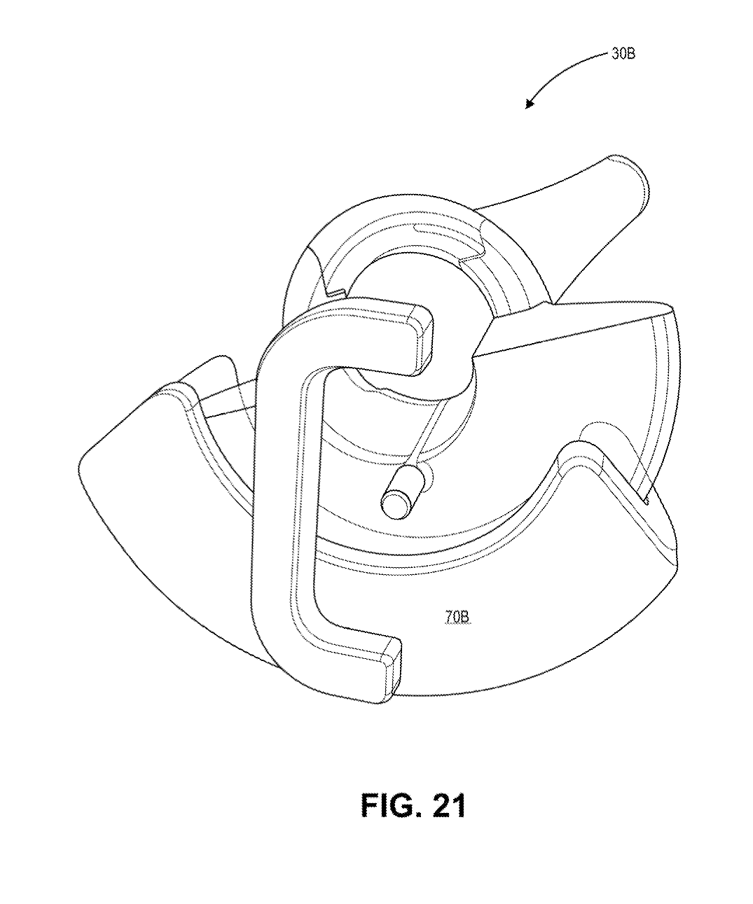

Unlike speculum 35, speculum 35b is configured to expand inside the orifice. The speculum is shaped to promote insertion into the vagina and gradually expand the orifice. As shown, for example, in FIG. 21, the exemplary speculum has distal tip with a relatively narrow diameter and the body flares extending towards the proximal end. Other shapes are contemplated including, but not limited to, a conical shape, a funnel shape, and a pyramidal shape. The distal tip can be easily inserted into the orifice. As the speculum is further inserted into the orifice, the flared sidewalls gradually expand the muscles surrounding the orifice. The gradual opening of the vaginal tract improves comfort because the muscles remain relaxed. Speculum 35b also includes magnification and illumination functions similar to speculum 35 described above.

Speculum 35b is configured to further expand the orifice. The exemplary speculum is configured to expand beyond the cervix to allow viewing of the uterus. The speculum body is configured in two pieces that open and close about a hinge at a proximal end (open position shown in FIG. 20). In an open position, the body is relatively narrower at a proximal end and relatively wider at a distal end. The smooth shape of the speculum and jaw opening function provides easier and more comfortable insertion into the vaginal tract. In various embodiments, the speculum is self-actuating so that it is pre-biased to an open position with springs or similar mechanisms. The speculum includes a mechanism for controlling opening of the body and locking the body in a selected open angle. The exemplary mechanism is a threaded pin that is adjusted by rotating the pin in a bore. The adjustment is carried out by rotating the pin so a tip of the pin extending beyond the base of the speculum and pushes open the body of the speculum. The pieces of the body are hinged together so pushing of one portion causes the other portions to also remain open.

The exemplary speculum 35b provides several benefits. The speculum can be easily operated by less experienced clinicians and increases patient comfort. The same speculum can be used to view the cervix and uterus. The cervix can be used by inserting the speculum to a position with the distal tip adjacent the cervix. The cervix can then be used in a similar manner to speculum 35 described above. The uterus can be viewed by opening the speculum body to expand the cervix and allow access to the uterus. Conventional systems require separate devices to view the cervix and uterus.

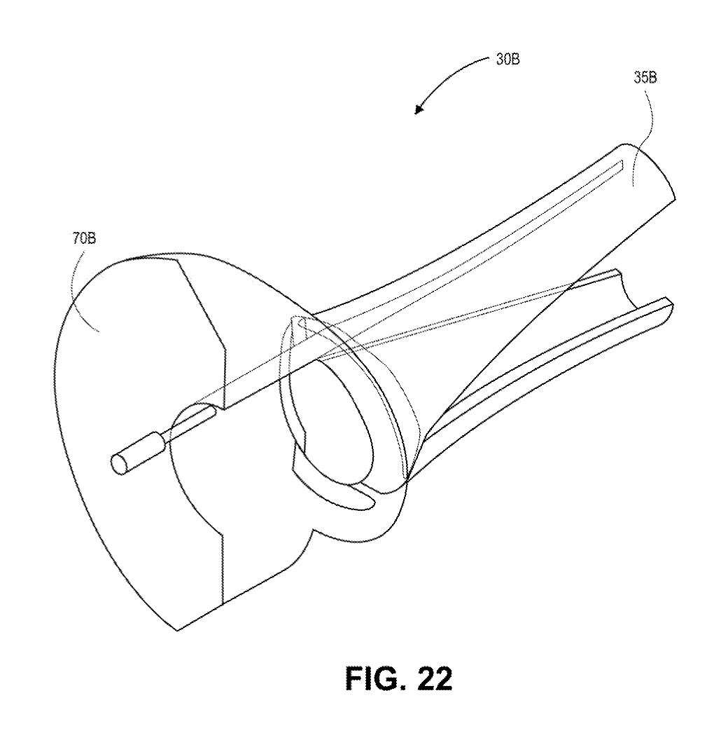

The exemplary device 30b is configured for remote screening processes. FIGS. 22-26 are sequential view of assembly of the device. Speculum body 35 is connected to a handle 70b. The handle is configured to allow snap attachment similar to described above. A remote device 80b is connected to the handle. The remote device includes a camera for making a recording of the subject being viewed. The remote device includes a communications system for transmitting information related to the captured image or data. The exemplary remote device is a smartphone. This allows the device to be used in developing countries and other environments with less readily-available resources.

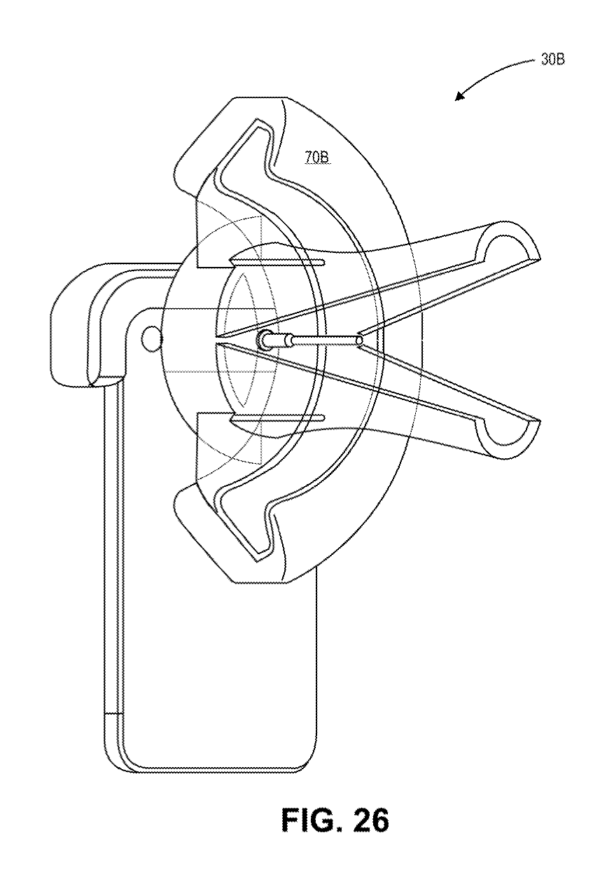

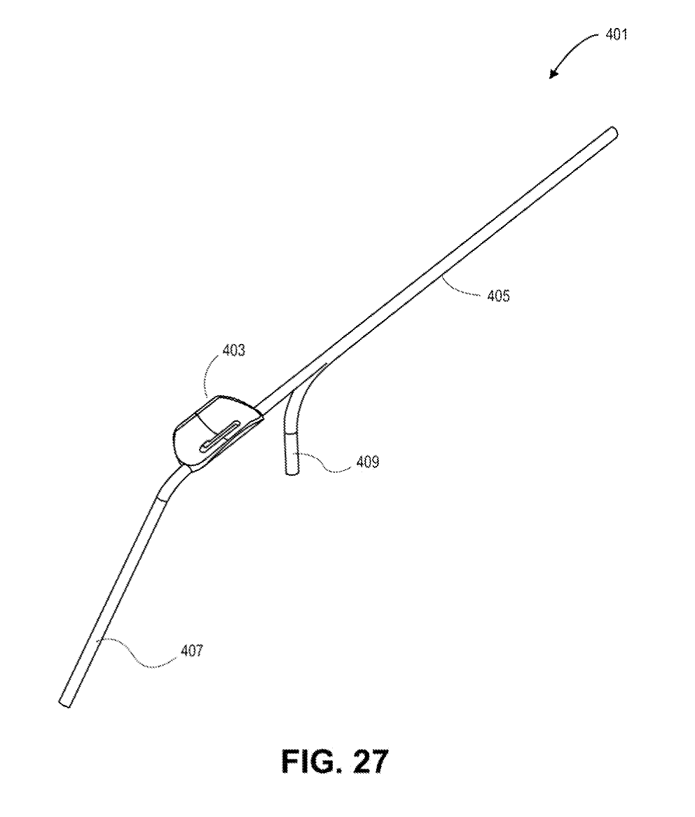

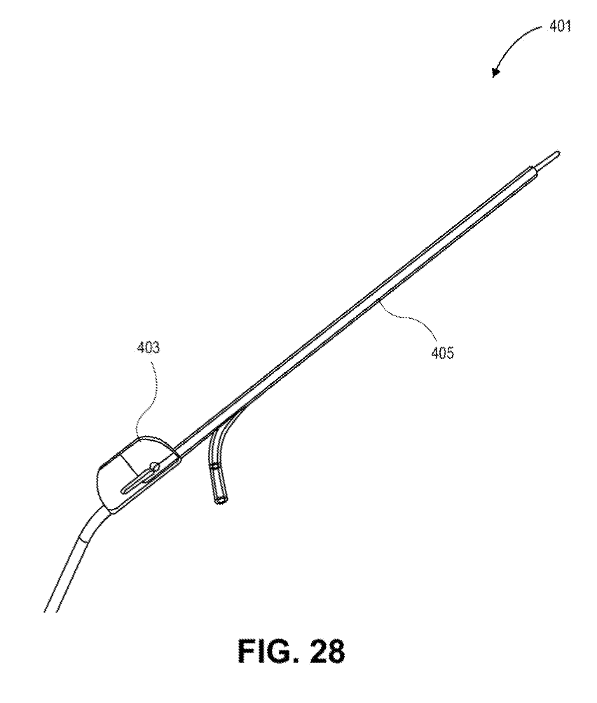

Attention is now directed to FIGS. 27 and 28 which illustrate a tool 401 for use with devices 30 and 30a described above. FIG. 27 shows the tool in a retracted storing position. FIG. 28 shows the tool in an extended working position with a working end of an instrument extending from the distal tool end. One will appreciate from the description herein that the devices enable use of a variety of existing and new tools. Tool 401 is one example of a modified tool for use with the devices. The exemplary tool improves ear wax removal.

Tool 401 includes several instruments. The tool combines suction, irrigation, and mechanical tool such as a curette tip. Typically a clinician must handle three separate tools for these functions.

The tool includes a housing 403 and an elongated body 405. The body may be shaped specifically for desired anatomy, e.g., the ear canal. The body can be conformable to the physiology.

A proximal end of the body includes a handle portion 407. A portion of the body defines a lumen therethrough. In the exemplary tool, the lumen extends from a distal most tip to the housing. The lumen is configured as a suction channel. An inlet 409 is in fluid communication with the inlet. The tool may include irrigation. In this case, the irrigation may be provided through inlet 409 or a separate inlet. The irrigation fluid may be delivered through the lumen. Alternatively, an auxiliary lumen may be provided in the main lumen but fluidly separate for this purpose.

The tool includes a curette instrument. The curette is similar to a conventional curette with a working end for removing ear wax. Other instruments may be provided including, but not limited to, forceps, a grasper, a cutting tool, a drug delivery tool, ablation device, and more. The curette is elongated and extends within the lumen. Suction and/or irrigation are provided around the curette body. In various embodiments, the instrument is configured to collapse and expand. For example, the instrument may be formed of a shape memory material so it collapses to a small shape when it is retracted and expands when axially extended out of the lumen.

The exemplary tool has three operational modes: tool, tool and suction, and suction. In the tool mode, the user can operate the curette tool. In the combined tool and suction mode, the tool is used while suction is available. This may be useful for performing biopsies, removing stenoses, clearing a path in impacted ear wax, and the like. In the suction mode, the tool can operate similar to a conventional suction device.

A proximal end of the curette is fixed in the housing with a stiff wire or similar fastener. The housing includes a slider control connected to the proximal end. The user can translate the curette with the slider. In operation, the curette is extended from the end of the lumen with the slider when the tool is needed. When the user is finished with the tool, the user slides the slider in a proximal direction so the curette is retracted and safely sheathed within the lumen. A plurality of tools may be provided through the lumen and selected with the handle controls as will be understood from the description herein.

One advantage of the tool described is that the user can view the treatment site even when the tool is deployed. The site is viewed through the lumen similar to the devices described above. Illumination may be provided through the walls of body 405 similar to the speculums described above. The tool also provides easy user access to several functional elements. The design is compact to allow use in even small body orifices. In various respects, the tool is similar to a catheter that includes suction. This reduces the need for the clinician and assistant to hold multiple tools. This also reduces cost.

In exemplary embodiment, the tool is configured for a myringotomy. In use, the tool is inserted into the body orifice (e.g. an ear canal). The tool may access the body orifice using device 30. For example, the distal end may be inserted through the bore in the speculum. The instrument (e.g. curette) is selected and the working end is extended to the work site. The exemplary instrument includes a cutting tool. An incision or hole is made in the ear drum. The cutting tool is retracted. Any excess material is removed through the suction channel. Alternatively, another tool, such as a grasper, may be provided to grasp and remove the material. A new prosthetic is then positioned in the hole using another instrument. The instrument is selected and then extended through the lumen. The prosthetic is placed. The instrument is then retracted. The entire tool is then removed from the orifice.

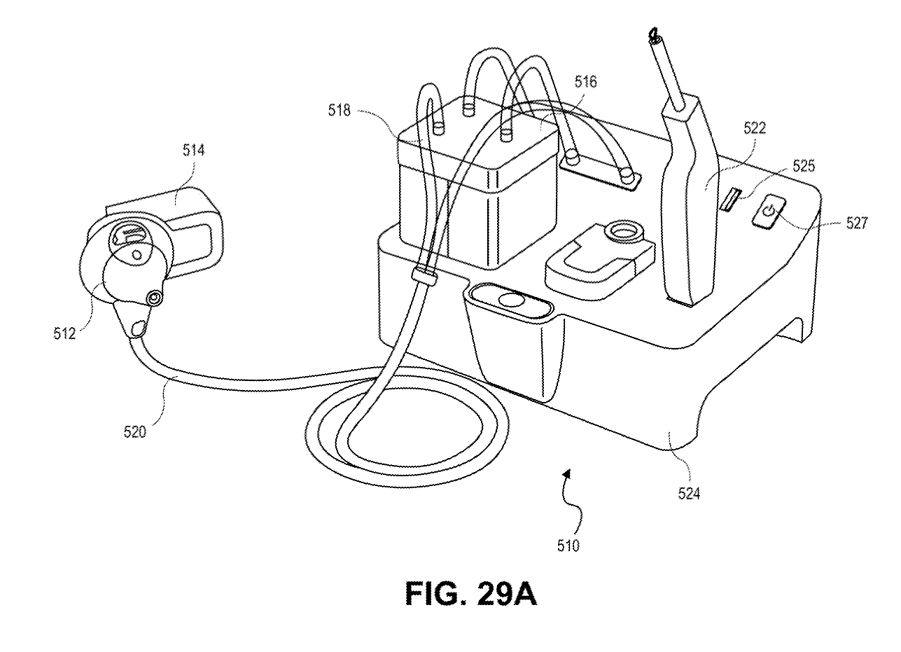

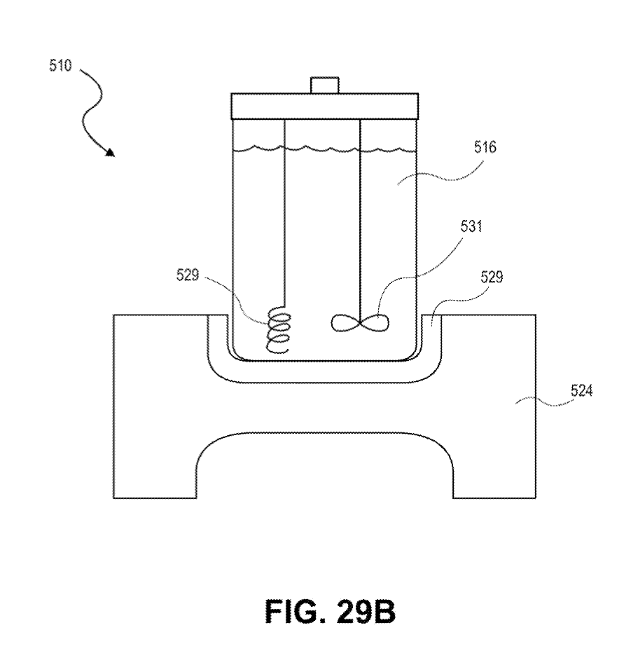

FIGS. 29-32 illustrate one embodiment of the system 510 or device for ear wax removal. As illustrated in FIG. 29A, the system 510 includes a speculum 512 that can be removably or reversibly attached to a handle adapter 514 and a source of irrigation such as a fluid reservoir 516 and vacuum 518 via a line or tube 520. In addition, the system 510 can include a suction curette 522 which is also attached to the vacuum source. A holder or housing 524 can be used to house the pump or pumps (not shown) and hold and organize for easy access one or more of the suction curette 522, fluid reservoir 516, the vacuum 518, handle adapter 514, speculum 512, and additional handle adapters and/or speculums and/or tubing. In some embodiments, the holder 524 can have a USB port 525 that allows the system 510 to be powered through a USB connection and can allow the system to be programmed and/or reprogrammed by the operator. For example, the USB connection can allow the operator to change the irrigation parameters, including flow rate, pulse rate and pressure of the irrigation fluid, for example. In some embodiments, the holder 524 can have a power switch 527 for turning the system on and off.

The system 510 can be designed to be compact and portable so that the operator can easily move the system 510 next to the patient. This increases the system's ease of use while also reducing material costs. For example, in some embodiments the holder 524 can be about 6 to 18 inches wide, 6 to 18 inches long, and 4 to 12 inches high, with the pump 524 sized to fit within the holder 524. In some embodiments, the holder 524 can be about 8 to 14 inches wide, 8 to 14 inches long, and 5 to 9 inches high. In some embodiments, the holder 524 can be about 11 inches wide, 11 inches long, and 7 inches high. In some embodiments, the system weighs between about 2 to 20 pounds, or about 2 to 10 pounds, or about 2 to 5 pounds, or less than about 25, 20, 15, 10, or 5 pounds.

In some embodiments as shown in FIG. 29B, the irrigation fluid can be heated to near or approximately body temperature, i.e. about 37 degrees Celsius, in order to reduce patient discomfort. Use of cold irrigation fluid within the ear canal may cause discomfort such as vertigo and nausea, for example. In some embodiments, the system 510 includes one or more heating elements 529. For example, the heating element 529 can be a hot plate on the holder 524 on which the fluid reservoir 516 rests. The hot plate can contact the bottom or bottom portion of the fluid reservoir 516. Alternatively or in addition, the heating element 529 can be a heating coil that is inserted into the fluid reservoir 516 to make direct contact with the irrigation fluid. The immersion type heating element 529 can also include a fluid circulator 531 that aids in mixing the fluid to promote even heating of the irrigation fluid. In some embodiments, the fluid reservoir 516 can include a fluid circulator 531 with or without an immersion type heating element 529 to provide mixing of irrigation fluid when the hot plate type heating element 529 is used.

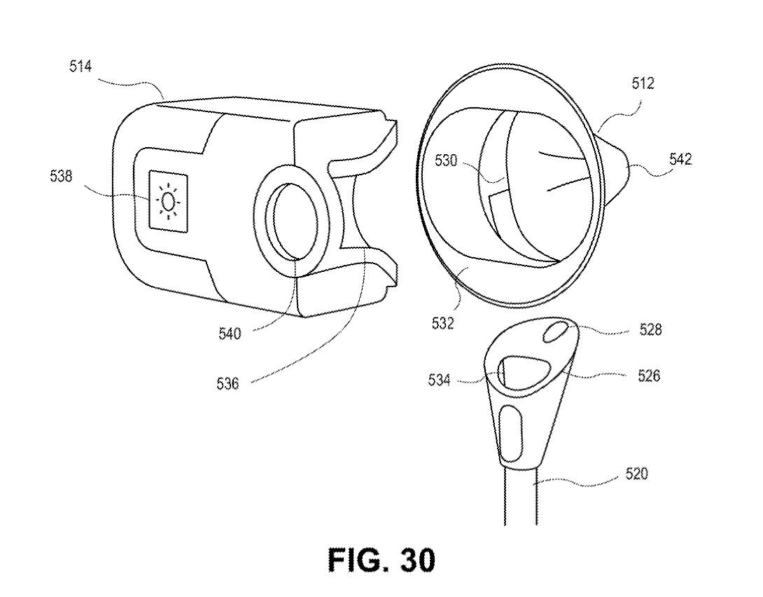

FIG. 30 shows the speculum 512, handle adapter 514 and tube 520 separated from each other and with additional details. The tube 520 can include both a vacuum line 526 and an irrigation line 528 that can be attached to the speculum 512. In some embodiments, the tube 520 can also include a wire in electrical communication with the PCB or microprocessor in the handle adapter 514 and the electrical circuits in the holder 524 that allow the pump or pumps to be controlled and allow the device to be powered. In some embodiments, the speculum can include an inner portion 530 that fits concentrically within the outer portion 532, forming irrigation channels between the inner portion 530 and outer portion 532. In some embodiments, when the tube 520 is connected with the speculum 512, the irrigation line 528 is in fluid connection with the irrigation channels between the inner portion 530 and outer portion 532 of the speculum 512, while the vacuum line 526 is in fluid communication with the interior space of the speculum 512. In some embodiments, the vacuum line is attached to the bottom portion of the speculum, which aids in the removal of irrigation fluid which tends to flow and/or pool at the bottom portion of the speculum due to gravity. In some embodiments, the vacuum line 526 has a tray or reservoir 534 to receive excess irrigation fluid in the event that the suction or removal of irrigation fluid is momentarily insufficient to remove all the irrigation fluid being introduced within the ear canal.

In some embodiments, the handle adapter 514 can be attached to the speculum 512 such that the handle adapter 514 extends from one side of the speculum 512 while the vacuum line 526 extends downwards. In other embodiments, the handle adapter 514 can be attached to the speculum 512 such that the handle adapter 514 extends downwards or upwards from the speculum 512 while the vacuum line 526 extends downwards. This configuration may provide ambidextrous use of the handle adapter.

FIG. 30 also illustrates that the handle adapter 514 includes a light source 536, which can be an LED for example, for illuminating the ear canal, a light source control switch 538 for turning the light source on and off, and a lens 540 for viewing into ear canal and through the handle adapter 514 and speculum 512. When the speculum 512 is assembled with the handle adapter 514, the lens 540 can be aligned with the distal opening 542 of the speculum 512.

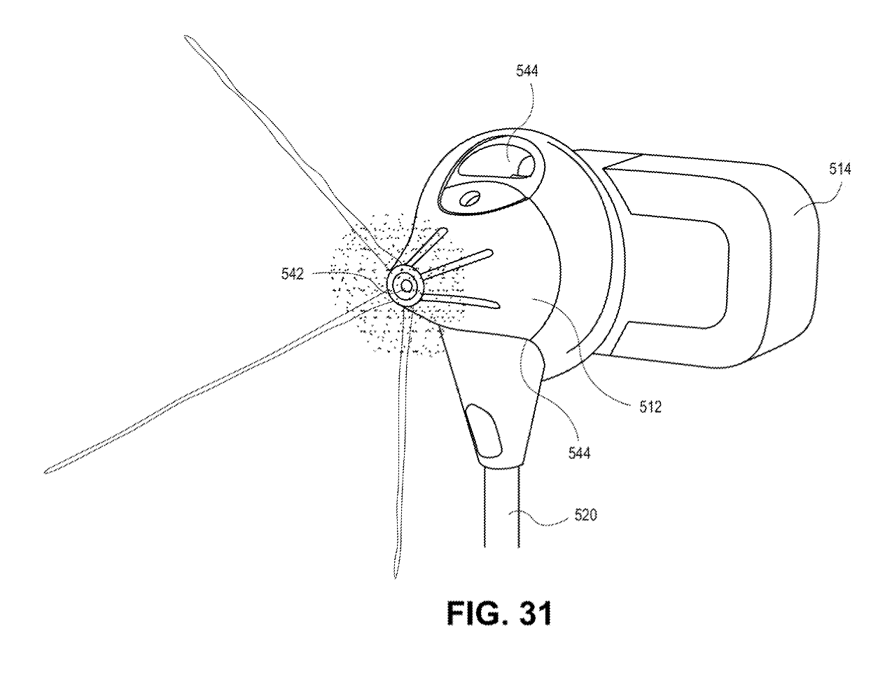

FIG. 31 illustrates the components shown in FIG. 30 after assembly. In some embodiments, the distal portion of the speculum 512 can have four exit ports that create four jets of water. In some embodiments, the speculum has two line ports 544 that connect the tube 520 to the speculum 512, where either line port 544 can serve as the bottom line port depending on the orientation of the speculum 512 and handle 514. In some embodiments, the proximal portion of the speculum 512 is open to the atmosphere even after the speculum 512 is introduced into the ear canal and a tight seal is formed. This proximal opening allows instruments to be inserted into the speculum during the ear wax removal procedure.

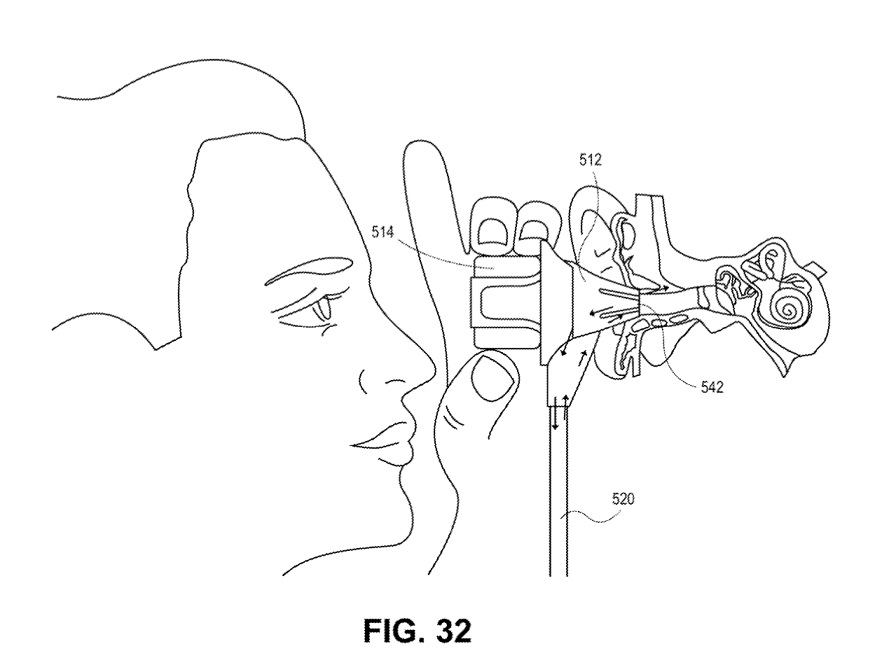

FIG. 32 illustrates the speculum 512 within a patient's ear canal with irrigation fluid being introduced to dislodge, break up and remove ear wax. As shown, the irrigation fluid can be ejected from the speculum at about 20 degrees from the ear canal axis, such that the irrigation fluid does not directly impinge upon the ear drum. In some embodiments, the irrigation fluid can be ejected at less than about 20 degrees, such as 15 degrees or 10 degrees, or less than 15, 10, or 5 degrees. In other embodiments, the irrigation fluid can be ejected at greater than 20 degrees, such as 25, 30, 35, 40, or 45 degrees. As shown in FIG. 32, the operator of the device can look through the lens and monitor the progress of the ear wax removal procedure. As ear wax is dislodged and flushed out with the irrigation fluid, the ear wax is sucked into the vacuum line and removed. The operator can also see if a large chunk or piece of ear wax obstructs the distal opening 542, which can prevent or impede the proper removal of irrigation fluid. In this situation, the operator can remove the speculum 512 from the ear canal and manually remove the ear wax from the device. In addition or alternatively, the operator can insert the suction curette 522 into the proximal opening of the speculum 512 in order to dislodge and/or remove the ear wax from the distal opening. In some embodiments, an additional sealing collar (not shown) can be inserted over the speculum 512 to improve the seal with the ear canal. The sealing collar can be made of a deformable material that can conform to the shape of the patient's ear canal.

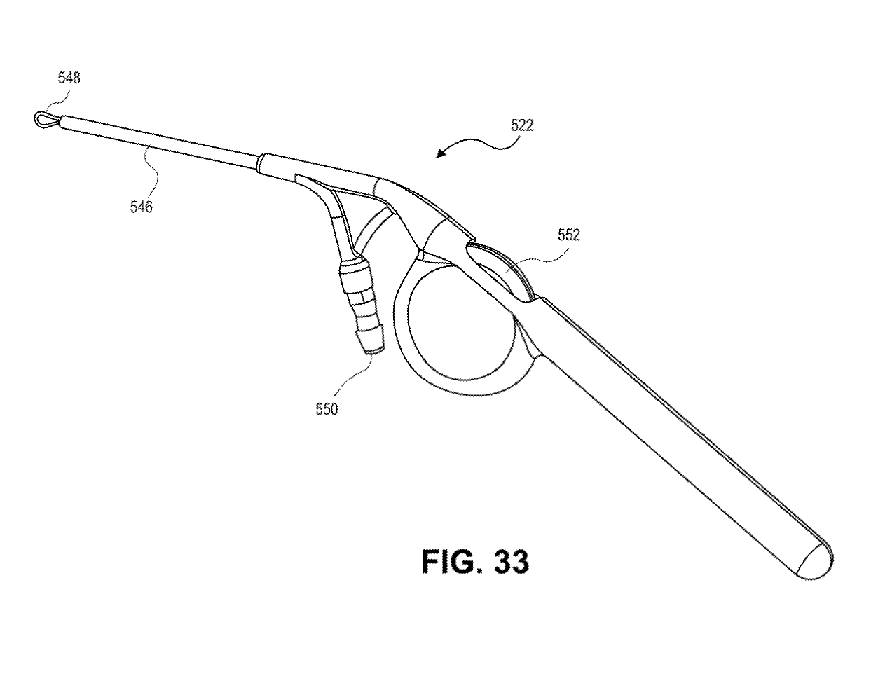

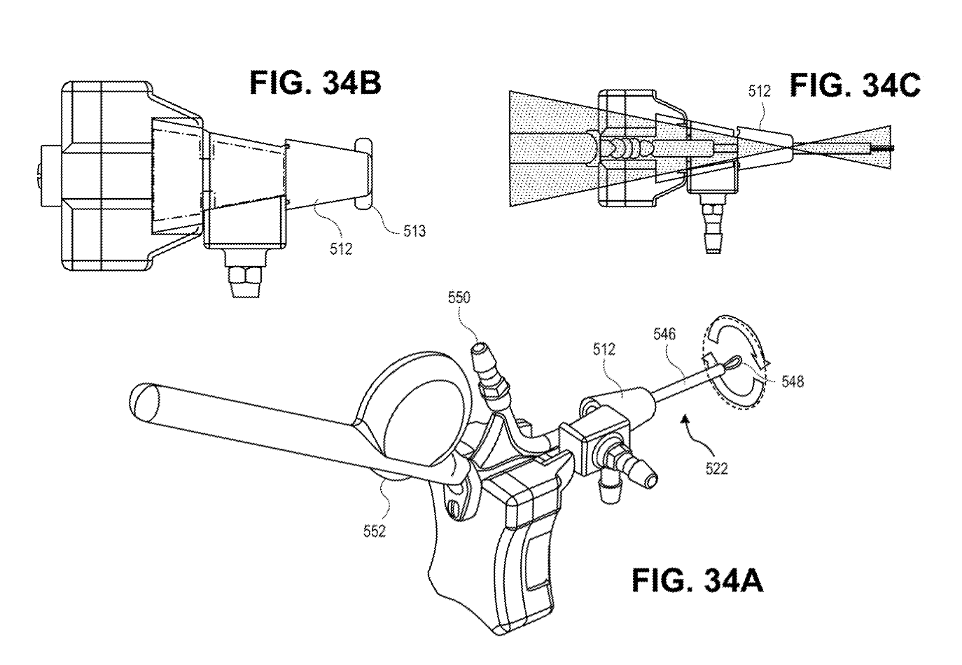

FIGS. 33 and 34A-34C illustrate an embodiment of a tool such as a suction curette 522. The suction curette 522 can include a tube or catheter 546 in which the curette 548 is disposed. The catheter 546 can be connected to a vacuum line via a vacuum line connector 550. The curette 548 can have a distal loop for dislodging and removing ear wax and can have an elongate body that is made of nitinol or another superelastic shape memory metal. Being made from a superelastic shape memory metal allows the curette to be repeatedly retracted and deployed from within the catheter using a retraction and/or deployment mechanism, such as a tool retraction/deployment wheel 552. The elongate body of the curette 548 can be wrapped around an axle that is rotated by the wheel 552. The wheel 552 can have a circumferential groove for facilitating the wrapping of the elongate body around the wheel 552. The operator can rotate the wheel 552 using, for example, a thumb or finger to retract and deploy the tool, such as a curette or myringotomy tool. This allows the operator to translate circular motion of the wrapped portion of the elongate body into linear motion of the distal portion of the tube, allowing the user to hold the tool in one place while retracting or deploying the device. In use, as the suction curette 522 is inserted through the proximal opening and through the distal opening of the speculum 512. The curette 548 is deployed to dislodge the ear wax, and the catheter 546 can be used to suck up the dislodged ear wax via the vacuum line. In some embodiments, the suction curette 522 is employed after determining that the fluid irrigation procedure alone using the speculum 512 is insufficient from removing all the ear wax. In other embodiments, the suction curette 522 can be used with the irrigation procedure or can be used instead of the irrigation procedure.

FIG. 34B illustrates an optional sealing element 513 than functions to provide a fluid tight seal between the speculum 512 and the ear canal when the speculum 512 is inserted into the ear canal. The sealing element 513 can be a ring of compressible and/or deformable material located on the distal portion or end of the speculum 512. The material can be reversibly compressible and/or deformable. The ring of compressible and/or deformable material can be made of a variety of materials, such as a sponge or foam or can be a ring structure filled with a gas such as air. As the speculum 512 is inserted into the ear canal, the sealing element 513 will conform to the geometry of the ear canal, thereby forming a fluid seal and preventing or reducing leakage of irrigation fluid from around the speculum 512 and out of the ear canal. In some embodiments the sealing element 513 is replaceable, allowing the operator to replace a worn sealing element 513 with a new sealing element 513, or allowing switching out a sealing element 513 with a first stiffness with a sealing element 513 with a second stiffness, wherein the first stiffness can be either less than or greater than the second stiffness, or allowing switching out a sealing element 513 made of a first material with a sealing element 513 made of a different material.

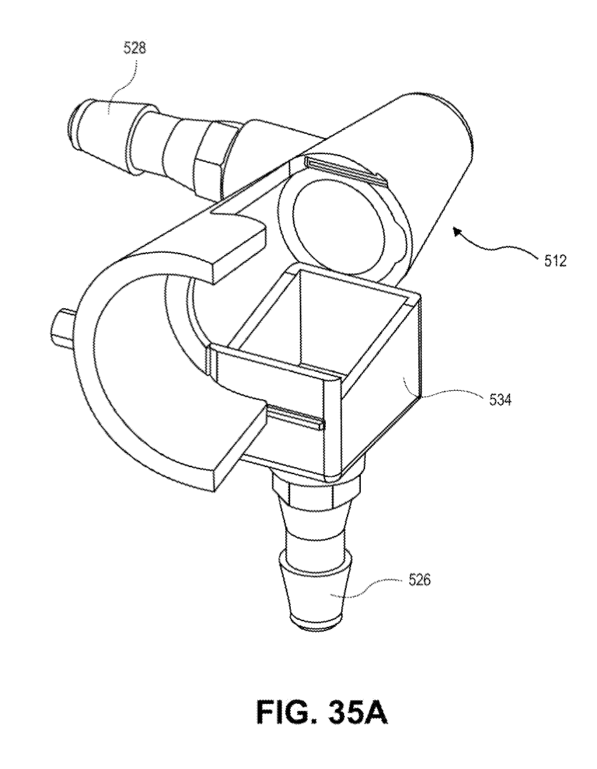

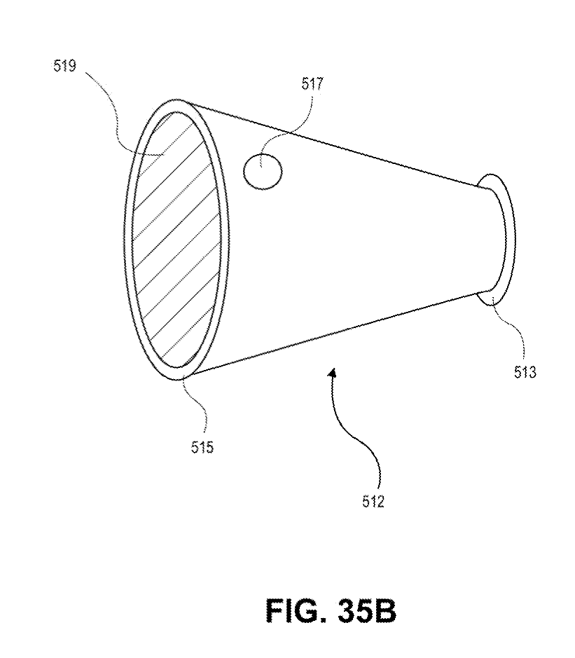

FIG. 35A illustrates another embodiment of the speculum 512 with separate connectors for the irrigation line 528 and the vacuum line 526. As illustrated, the vacuum line 526 is connected to the bottom of the speculum 512 with a tray or reservoir 534 to receive and hold irrigation fluid and to prevent or reduce overflow of excess irrigation fluid. In addition, as illustrated, the speculum 512 has an open design where the proximal end of the speculum 512 is open and exposed to the atmosphere. This open design allows tools to be inserted through the speculum and allows the pressure within the ear canal to remain at or around atmospheric pressure after insertion of the speculum 512 and during the ear wax removal procedure.

FIG. 35B illustrates an embodiment of a closed speculum design, where the proximal end 515 of the speculum 512 can be closed to or sealed from the atmosphere. A closed speculum design can allow a negative pressure or vacuum to be developed within the speculum and ear canal if desired. A negative pressure can be desirable for sucking a large ball of ear wax that is clogging the distal end of the speculum into the speculum 512. A sealing element 513 as described herein can be placed on the distal end of the speculum to facilitate sealing the ear canal to further facilitate development of the vacuum within the speculum 512. The speculum 512 can have a vacuum relief port 517 located on, for example, the sides or top of the speculum 512. The vacuum relief port 517 provides communication between the interior of the speculum 512 and the atmosphere. While the vacuum relief port 517 remains open or unblocked, a vacuum or negative pressure will not develop within the speculum 512, and the speculum 512 can be considered to be in an open configuration. However, the operator can removable or reversibly close or block the vacuum relief port 517, by for example covering the vacuum relief port 517 with a thumb, finger, removable plug, removable patch or other material, when generating a negative pressure or vacuum within the speculum is desired. When the vacuum relief port 517 is closed, the speculum 512 is in a closed configuration and can develop a vacuum or negative pressure from the action of the vacuum line. By removing the blockage from the vacuum relief port 517, the speculum 512 can be restored to an open configuration at atmospheric pressure. In some embodiments, the negative pressure or vacuum generated within the speculum by the vacuum line within the speculum can be up to about 24.5 inches of Hg. In some embodiments, the negative pressure or vacuum generated by the vacuum line within the speculum can be less than or greater than about 24.5 inches of Hg. In some embodiments, a lens 519 can be used to close or seal the proximal end of the speculum 512, allowing for improved magnification within the ear canal when used in conjunction with the lens on the handle adaptor.

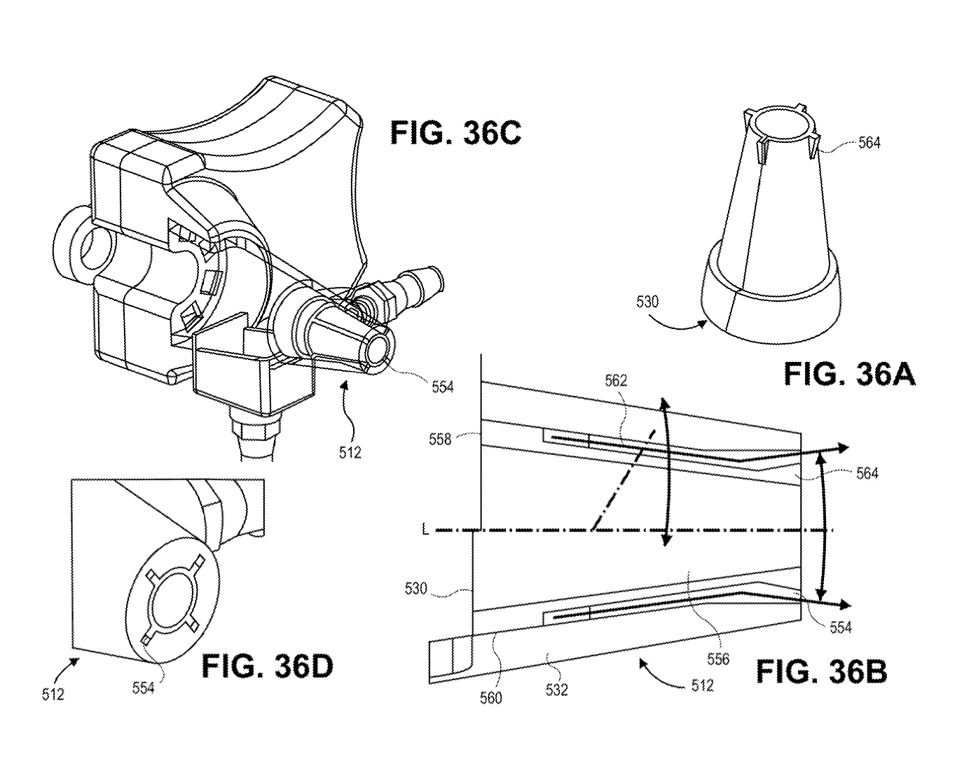

FIGS. 36A-36D illustrate an embodiment of the speculum 512 with four exit ports 554. The distal portion 556 of the inner portion 530 of the speculum can have material removed such that the proximal portion 558 of the inner portion 530 forms a seal 560 with the outer portion 532 while leaving a gap or channel 562 between the distal portion 556 and the outer portion 532. In some embodiments, the channels can be straight and/or substantially occupy the distal portion of the speculum 512 in order to minimize or reduce the pressure drop in the irrigation fluid. The inner portion 530 can include four fluid guides 564 that control the angle of the irrigation fluid that exits the exit ports 554. For example, the fluid guides 564 can be angled at 20 degrees from the longitudinal axis L of the speculum, which results in fluid exiting the exit portions at 20 degrees from the longitudinal axis. The fluid guides 564 can be angled at less than about 20 degrees, such as 15 degrees or 10 degrees, or less than 15, 10, or 5 degrees. In other embodiments, the fluid guides 564 can be angled at greater than 20 degrees, such as 25, 30, 35, 40, or 45 degrees. In some embodiments, an inner portion with four fluid guides 564 is preferable over three fluid guides because a four fluid guides 564 embodiment can be more easily manufactured than a three fluid guides embodiment in some molding processes. The four fluid guide 564 embodiment results in a mold configuration that is easier to pull off of the inner portion 530. In other embodiments, the inner portion 530 can have less than 4 fluid guides, such as 1, 2 or 3 fluid guides. In other embodiments, the inner portion 530 can have greater than 4 fluid guides, such as 5, 6, 7 or 8 fluid guides, for example.

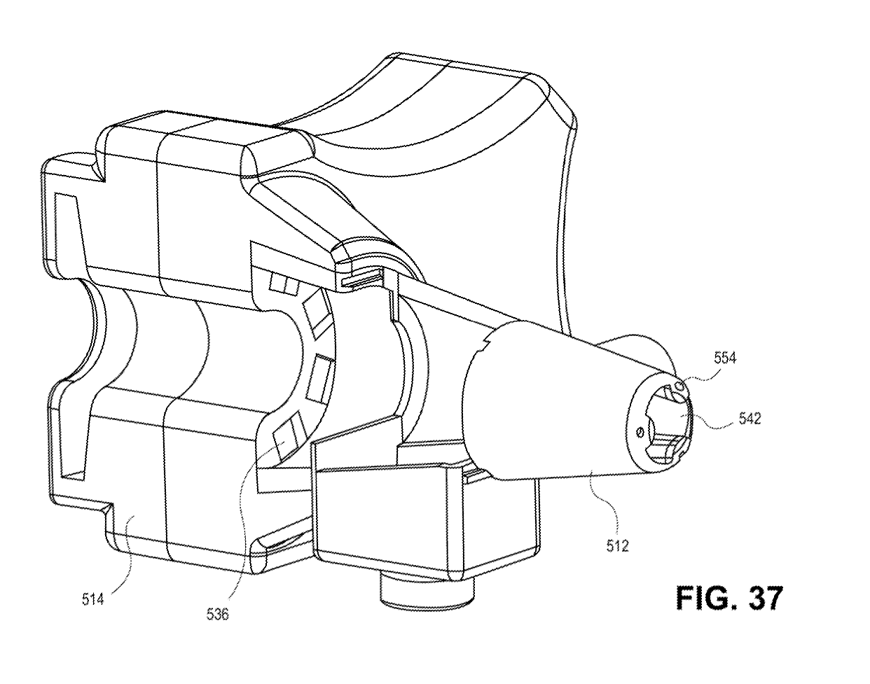

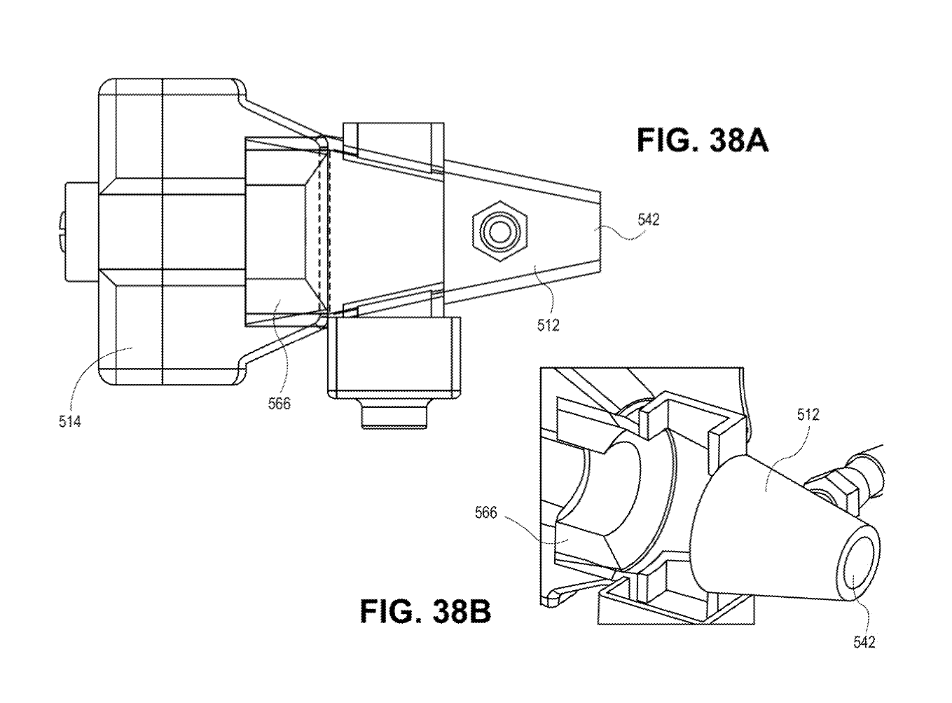

FIGS. 37-38B illustrate an embodiment of the lighting mechanism used by the system. As illustrated in FIG. 37, a plurality of light sources 536, which can be LEDs, can be arranged on the handle adapter 514 to project light into the speculum 512. As illustrated in FIGS. 38A and 38B, a light pipe 566 can be inserted between the light sources 536 and distal opening 542 of the speculum 512. The light pipe 566 is optically translucent and can function to focus the light through the distal opening 542 of the speculum to improve illumination of the ear canal. In some embodiments, the light pipe 566 is made of acrylic, polycarbonate, or some other optically clear polymer. In other embodiments, the light pipe 566 can be made of a nonpolymeric material such as glass. In some embodiments, without the light pipe 566 the light from the light sources 536 is scattered by reflections within the structures of the speculum 512, resulting in dimmer illumination of the ear canal.

FIG. 37 also illustrates an embodiment of the distal opening 542 where the size of the opening is increased or maximized to the extent possible, where the distal opening 542 bends around the exit ports 554 rather than simply being a circle that is inscribed within the exit ports 554. Increasing the size of the distal opening reduces the likelihood that a piece of ear wax will block the distal opening 542 and prevent or interfere with suctioning and removal of the irrigation fluid from the ear canal.

In some embodiments, a foot pedal (not shown) can be used to control the pressure and/or flow rate of the irrigation fluid. In some embodiments, depressing the foot pedal can increase the pressure and/or flow rate within a predetermined or preselected range, up to a maximum predetermined value to ensure patient safety.

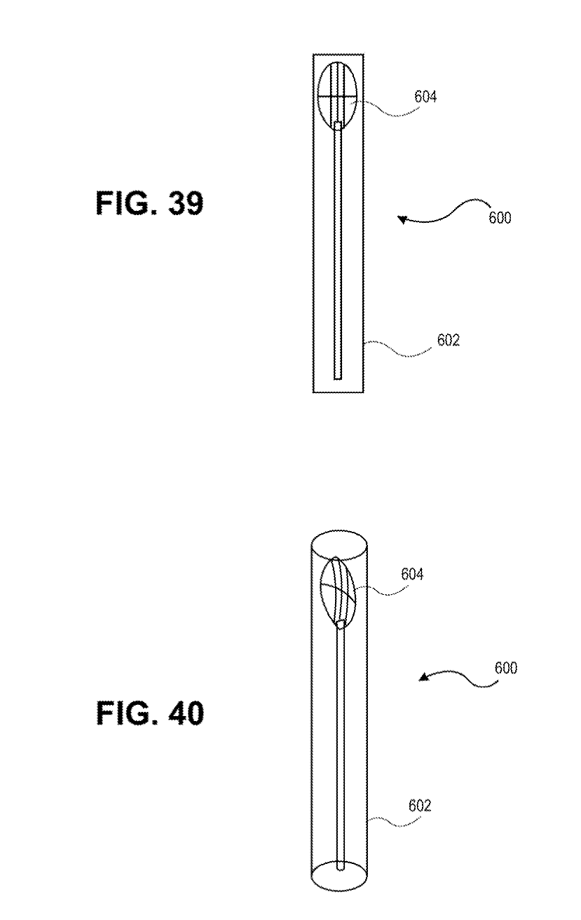

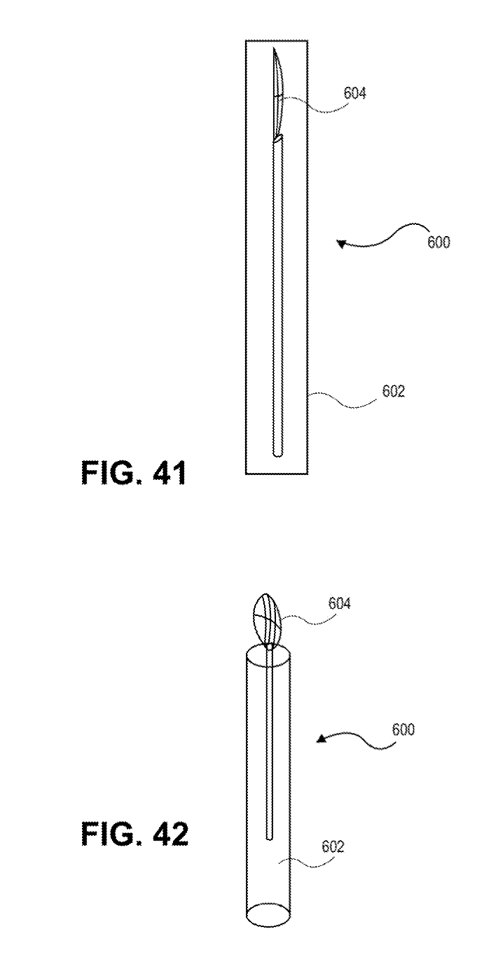

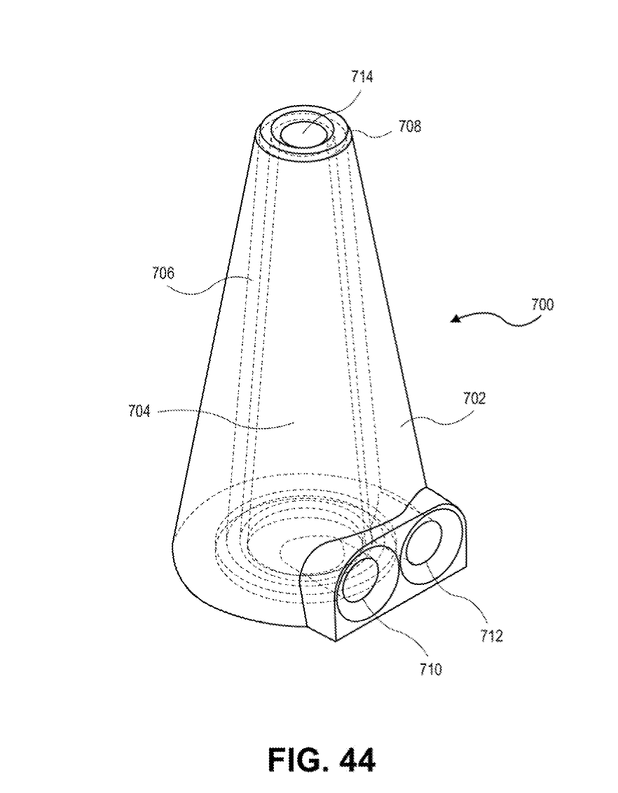

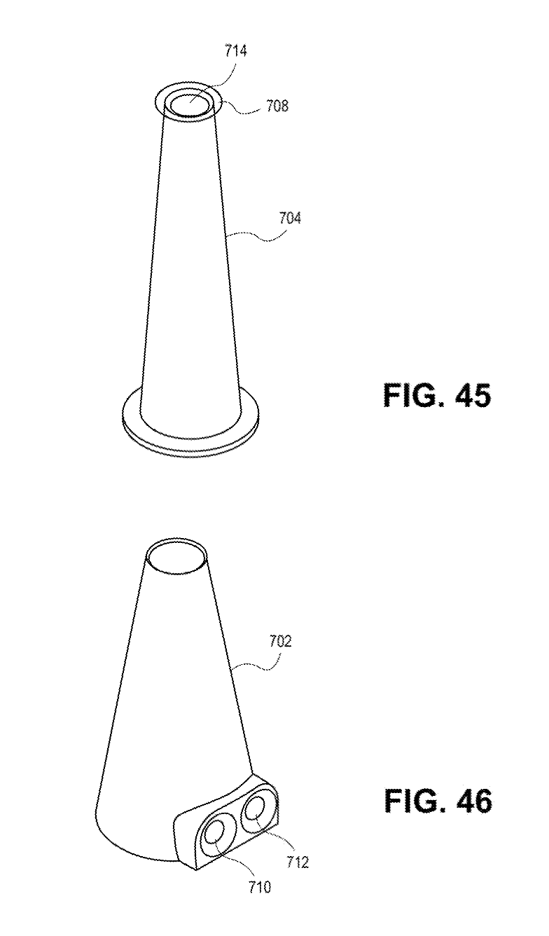

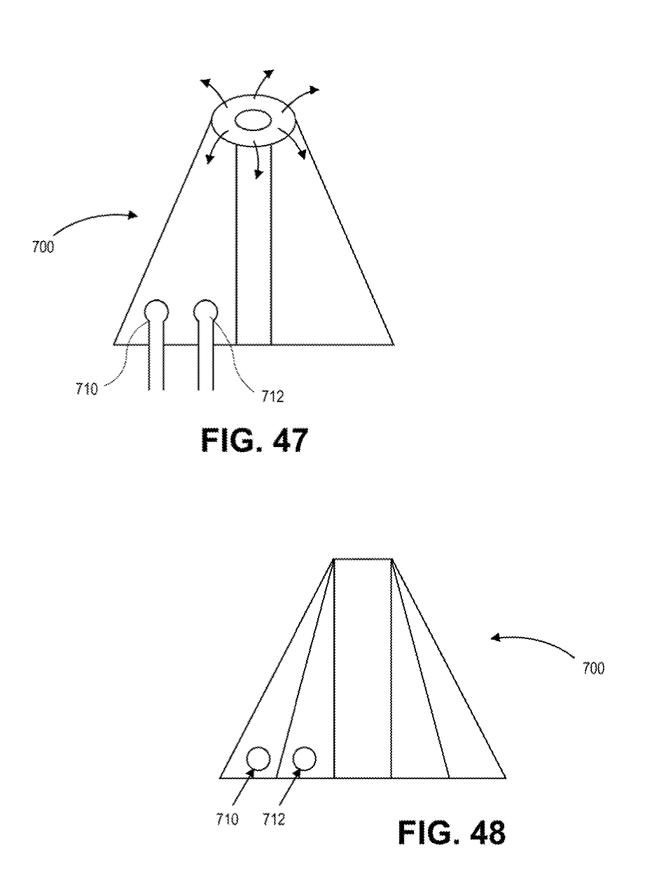

FIGS. 39-48 illustrate other embodiments of an ear wax removal device. These devices provide a fast, safe, effective way to remove earwax, especially the impacted type, from the ear canal to reduce ear wax-related hearing loss, alleviate patient discomfort, and/or prep for an audiology exam.

FIGS. 39-42 illustrate an embodiment of an ear wax removal device 600 comprised of a tube 602 for irrigation or suction, an instrument 604, such as a curette, integrated or disposed within the tube 602, an actuator for scalable movement and retraction of the instrument 604, and an attachable suction or irrigation apparatus.

The device 600 has a tube 602 for irrigation or suction with integrated retractable and scalable instruments 604, enabling switching between suction only, suction and instrumentation simultaneously, and instrumentation without suction. In one variation the instrument 604 is curette. In another variation, the instrument is a blade. In another variation, the instrument is a forceps. In another variation, the instrument is scissors. In another variation, the instrument is an irrigation or suction tube. In another variation, the instrument is a camera. In another variation, the instrument is a cauterizer. The material for the instrument 604 can be made of an elastic material that is capable of collapsing and then expanding to the desired instrument shape once actuated. One such material is nitinol. The actuator allows the instrument 604 to be actuated when use is desired and then retracted when only suction (no instrumentation) is desired. In one variation, the actuator is a rack and pinion mechanism. In another variation, the actuator is a sliding mechanism. The attachable suction or irrigation apparatus acts as the source of vacuum or water. In one variation, the suction apparatus is a one in which mechanical vacuum is created through springs that's move a piston back and forth. In a second variation, the suction/irrigation apparatus is a motor powered vacuum/pressure pump. In another variation, the suction/irrigation apparatus is created using a venturi system.

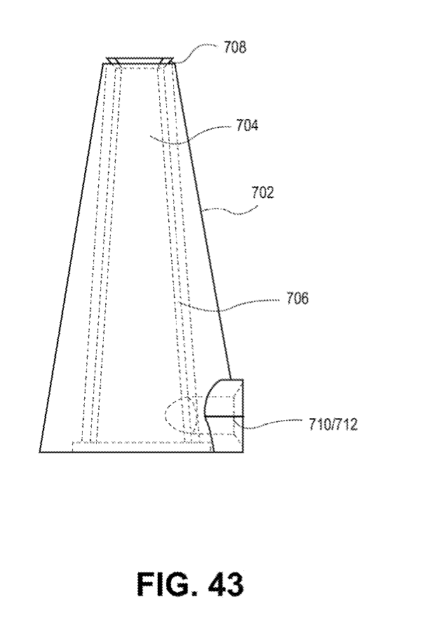

FIGS. 43-48 illustrate another embodiment of an ear wax removal device 700 comprising an outer ear speculum 702 to straighten the ear canal or for insertion into the ear canal, and an inner speculum 704 with a flared tip 708 to deliver a 360 degree ring of water stream directed at the ear canal wall, and an attachable irrigation/suction apparatus.

The device can be a nested speculum that provides a safer way to irrigate an ear. The device can be made of two generally funnel-shaped members, an outer ear speculum 702 and an inner speculum 704 for example, nesting together. There is a channel 706 created for irrigation/suction between the nested specula pieces. The channel 706 can be created by sizing the inner diameter of the outer speculum 702 to be greater than the outer diameter of the inner speculum 704. The channel 706 can be cone shaped or tube shaped. When the speculum 700 is inserted into the ear, it provides an effective way of irrigating the ear canal.

Rather than a focused beam of water entering the ear canal, the nested speculum 700 can be configured to deliver a spiral or conical or angled water stream or streams instead of a straight jet stream into the ear canal. The stream of water from the nested speculum 700 can have a lower pressure than the tradition jet stream from traditional irrigation devices because the water stream from the nested speculum 700 can be diffused. Furthermore, the water stream can be configured to come out as full ring or cone, providing irrigation over 360 degrees and can be directed out at an angle towards the ear canal walls rather than directly at the ear drum. In contrast, a traditional high powered jet stream that directly impacts the ear drum can sometimes cause complications such as a perforated eardrum. The delivery technique using the nested speculum 700 can reduce the complication rate associated with irrigation. In one variation, the flared tip 708 is created by a flared notch so when the water hits it, it is directed out at an angle towards the wall of the ear canal.

In some embodiments, the speculum 700 has ports, such as a suction port 710 and an irrigation port 712, for attachment of a suction and irrigation apparatus. In some embodiments, the irrigation port 712 can be in fluid communication with the channel 706. In some embodiments, the suction port 712 can be also in fluid communication with the channel 706. In some embodiments, the suction port 712 can be in fluid communication with an inner channel 714 of the inner speculum. Once connected to the speculum 700, the suction and irrigation apparatus can provide alternating or simultaneous irrigation and suction. The speculum 700 can deliver a ring of irrigation fluid, followed by suctioning back the irrigation fluid and removed or dislodged ear wax. This method of removing ear wax can be more effective than traditional methods. In another embodiment, the speculum 700 can be made of 3 nested parts, thereby creating separated rings for irrigation and suction. Alternatively, in a speculum 700 made of two nested parts, the channel 706 formed between the outer speculum 702 and inner speculum 704 can be used for irrigation and can be connected and be in fluid communication with the irrigation portion 712, and the inner speculum 704 can have an inner channel 714 that can be used for suction and can be connected and be in fluid communication with the suction port 710. In this variation, the speculum 700 could be connected to distinct suction and irrigation apparatus/sources and because they are operating through separate channels, simultaneous irrigation and suction can be used during the ear wax removal procedure.

The attachable suction or irrigation apparatus acts as the source of vacuum and/or irrigation fluid such as water. In one variation, the suction apparatus is a one in which mechanical vacuum is created through springs that's move a piston back and forth. In a second variation, the suction/irrigation apparatus is a motor powered vacuum/pressure pump. In another variation, the suction/irrigation apparatus is created using a venturi system.

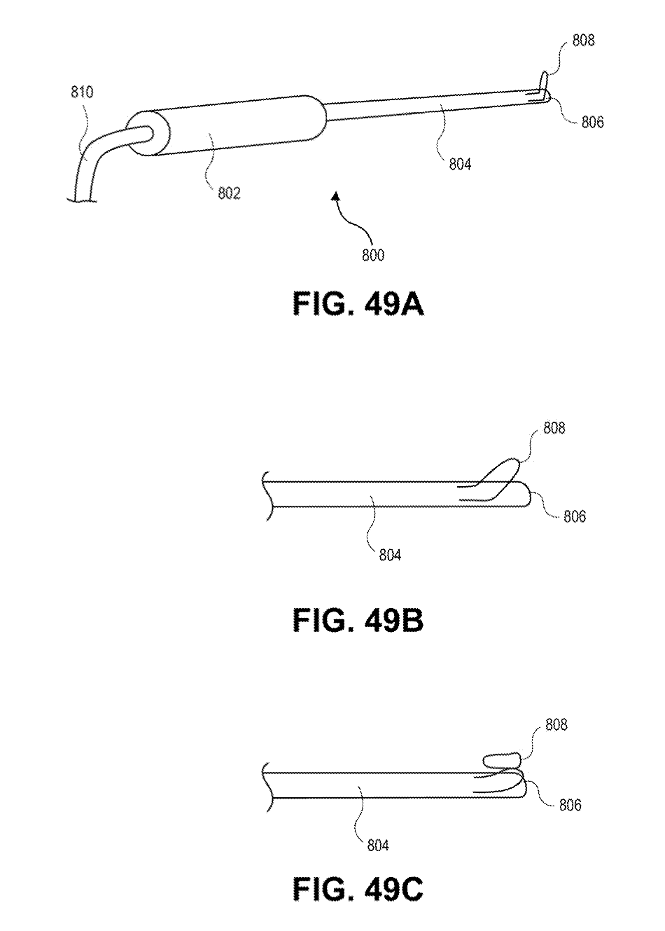

FIGS. 49A-49C illustrate other embodiments of the suction curette illustrated in FIGS. 33 and 34. In FIG. 49A, the suction curette 800 has a handle portion 802 that facilitates gripping and handling of the suction curette, a suction tube 804 with a suction lumen with a opening at the distal end 806 of the suction tube 804, and a wire loop 808 that does not extend past the distal end 806 of the suction tube 804. Keeping the wire loop 808 proximal the distal end 806 facilitates the suctioning of ear wax by the suction tube 804 and reduces the likelihood of pushing the ear wax distally away from the suction tube 804 during the ear wax removal process. In some embodiments, the wire loop 808 can be attached to the outer surface of the suction tube 804. In some embodiments, the wire loop 808 can extend radially and perpendicularly away from the surface of the suction tube 804 as shown in FIG. 49A. In other embodiments, as shown in FIGS. 49B, the wire loop 808 can be angled with respect to the surface of the suction tube 804. In some embodiments, the angle can be between 15 degrees and 75 degrees, or between about 30 degrees and 60 degrees, with the wire loop 808 extending in the distal direction. In other embodiments, as illustrated in FIG. 49C, the wire loop 808 can be configured to extend distally and then hook backwards in the proximal direction to form a hooked loop configuration. In these embodiments, the wire loop 808 can be fixed rather than extendable and retractable. In other embodiments, the wire loop 808 can be made to be extendable and retractable, but can be also made to not extend past the distal end 806 of the suction tube 804. In some embodiments, the wire loop 808 can have a stiffness that allows it to dislodge and/or break up ear wax but not penetrate through the ear drum. In some embodiments, the handle portion 802 can be shaped like a pencil or pen grip, and can be circular, triangular, or have another cross-sectional shape that is ergonomic to hold. The handle portion 802 can be made of a variety of materials, such as a rubber or relatively soft polymer that is tacky and/or non-slip and is easily gripped.

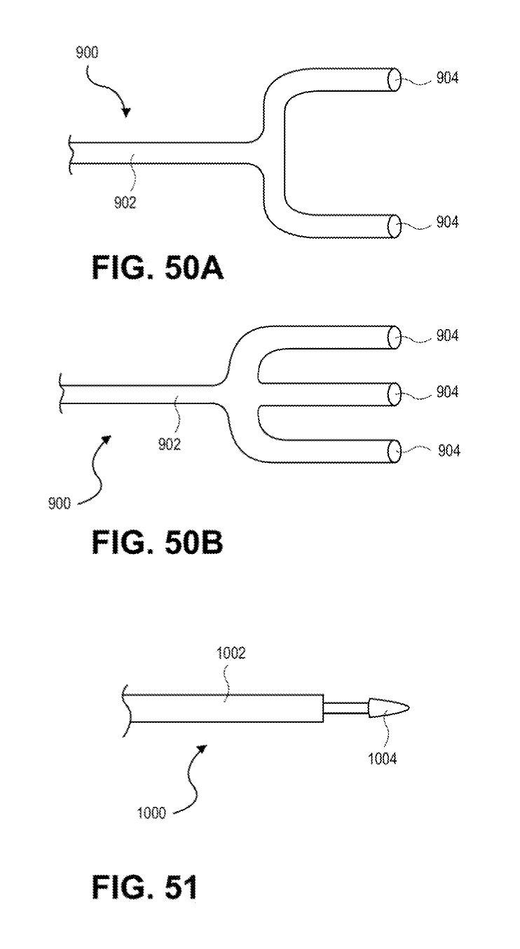

FIGS. 50A and 50B illustrate embodiments of modified suction tube 900 that can be used with a suction source, such as the suction sources described herein, to remove ear wax particles that clog ports in a device inserted into the ear canal, such as a hearing aid, an in-ear reference monitor or ear phone, or other in-ear device. The modified suction tube 900 can have a proximal tube portion 902 that can be connected to a vacuum source. The proximal rube portion 902 can branch out into one or more distal suction tube portions 904 that can be sized, arranged and configured to fit into or around the ports of the in-ear device or some other device. In some embodiments, the modified suction tube 900 can have two, three, or more than three distal suction tube portions 904. Each distal suction tube portion 904 can provide suction to such out ear wax debris or other debris from the ports or channels of the in-ear device.

FIG. 51 illustrates an embodiment of a curette 1000 with an extendable and retractable cutting tool 1004 that can extend from a tube or suction tube 1002. The cutting tool 1004 can be a wire loop with a sharpened distal tip portion. The distal portion of the wire loop can be sharpened to provide a cutting edge and a piercing tip. Alternatively, a blade can be attached to the wire loop or a shaft. The cutting tool 1004 can be used to perform a surgical procedure within the ear canal, such as a myringotomy in which an incision is made in the eardrum.

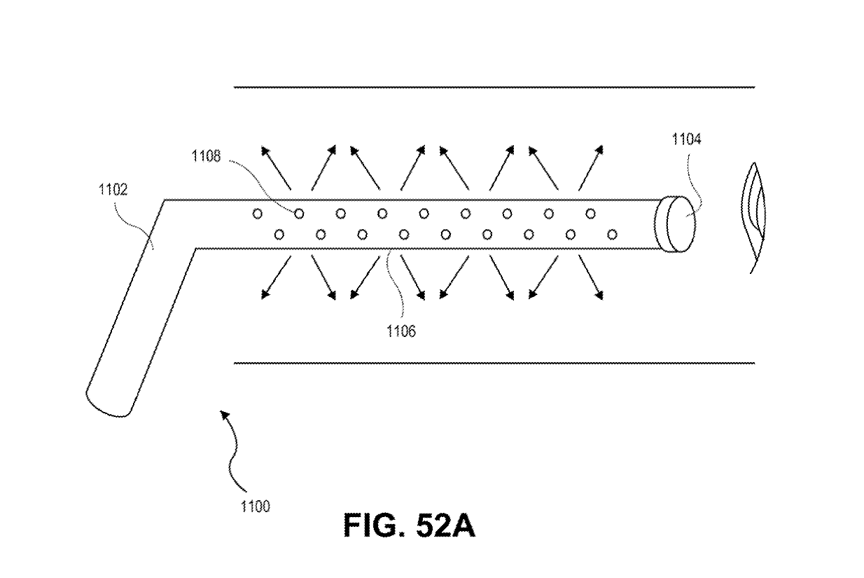

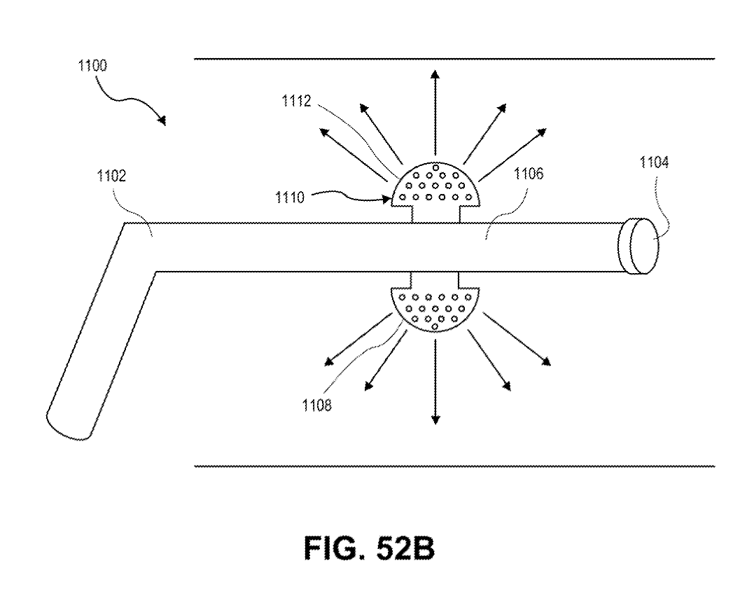

FIG. 52A illustrates an embodiment of a fluid delivery tool 1100 configured to provide fluid delivery to the ear canal. The tool 1100 has a hollow elongate body 1102, such as a tube or catheter that can be rigid, semi-rigid, or flexible. The elongate body 1102 can have a capped or sealed distal end 1104. The distal portion 1106 of the tool 1100 can include a plurality of holes or ports 1108. The proximal end of the tool 1100 can be connected to a source of fluid. In use, the fluid delivery tool 1100 is inserted into the ear canal to remove wax buildup around the walls of the ear canal. Irrigation fluid can be jetted out of the holes or ports 1108 in a radial direction towards the ear canal walls to dislodge and remove wax buildup on the walls. The fluid delivery tool 1100 can be used when a relatively small amount of ear wax remains adhered to the walls of the ear canal. The capped distal end 1104 prevents fluid from jetting out of the tool and directly impinging the ear drum.

FIG. 52B illustrates another embodiment of a fluid delivery tool 1100 with a hollow elongate body 1102, a capped distal end 1104, and a plurality of holes or ports 1108 for fluid delivery to the ear canal. In this embodiment, however, the holes or ports 1108 are located on one or more plugs or protrusions or blocks 1110 that extend radially from the distal portion 1106 of the elongate body 1102. In some embodiments, the plugs 1110 can have a curved portion 1112 where the holes or ports 1108 are located. The plugs 1110 can allow greater and/or more even jets of fluid from the ports by being located at about the same longitudinal portion of the elongate body 1102 and/or by allowing the number of holes or ports 1108 to be reduced. The curved portion 1112 also allows the holes or ports 1108 to be oriented in a plurality of directions, with some holes or ports 1108 being distally facing, some holes or ports 1108 being proximally facing, and some holes or ports 1108 being radially or transversely facing. In general, the holes or ports 1108 are oriented towards the wall of the ear canal. As illustrated, the tool 1100 has two plugs 1110, but in other embodiments, the tool 1100 can have one or more plugs 1110, such as 1, 2, 3, 4, 5, or 6 or more plugs 1110.

The fluid delivery tool 1100 embodiments can be particularly useful for removing ear wax from on the wall of the ear canal when placing and/or fitting a hearing aid into the ear canal. The hearing aid is typically placed close to the ear drum, for example within about 4 mm from the ear drum, and residual ear wax on the wall can plug up the ports on the hearing aid and/or interfere with the fit of the hearing aid within the ear canal.

In addition, it may be desirable to dry the ear canal after irrigation. This can be desirable for hearing aid fitting, where moisture can interfere with fitting the device and/or interfere with the hearing device itself. Air can be introduced into the ear canal using a variety of techniques. For example, the vacuum line and pump can be run in reverse to introduce air via the central bore. Alternatively, air can be introduced through the fluid channels between the inner and outer speculum instead of irrigation fluid.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. For example, one or more features of one particular embodiment can be combined with one or more features from another embodiment. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. In addition, the terms about and/or approximately can mean, for example, within 10%, within 20% or within 30%.

What is claimed is:

1. A device for accessing a body orifice, comprising:
an outer shell having a proximal end, a distal end, and a central bore;
an inner shell coaxial with the outer shell; and
one or more fluid channels defined between an outer surface of the inner shell and the inner surface of the outer shell.

2. The device of claim 1, wherein the device includes a suction channel.

3. The device of claim 1, wherein the outer shell comprises a body including an integrated illumination pathway.

4. The device of claim 3, wherein the integrated illumination pathway includes a light pipe.

5. The device of claim 1, wherein the inner shell comprises a central bore configured as a suction channel.

6. The device of claim 1, wherein the inner shell comprises a central bore configured to provide access to the body orifice for a medical instrument.

7. The device of claim 6, wherein the medical instrument is a curette.

8. The device of claim 1, wherein the device is configured for removal of cerumen from an ear canal.

9. The device of claim 1, wherein the inner shell has a longitudinal axis, a proximal end and a distal end, the distal end having one or more fluid guides configured to direct fluid at a predetermined angle away from the longitudinal axis.

10. The device of claim 9, wherein the one or more fluid guides is an even number of fluid guides.

11. The device of claim 9, wherein each of the one or more fluid guides has a fluid guide surface that is angled at a predetermined angle with respect to the longitudinal axis.

12. The device of claim 1, wherein the inner shell comprises a first outer surface and a second outer surface, wherein the second outer surface is offset from the first outer surface such that when the first outer surface of the inner shell is in contact with the inner surface of the outer shell, the second outer surface of the inner shell is offset from the inner surface of the outer shell, thereby forming the one or more fluid channels between the second outer surface of the inner shell and the inner surface of the outer shell.

13. The device of claim 1, wherein the one or more fluid channels are in fluid communication with a source of fluid.

14. The device of claim 1, wherein the outer shell has a sealing element located on a distal end of the outer shell, the sealing element configured to provide a fluid seal between the device and the body orifice.

15. The device of claim 14, wherein the sealing element is reversibly deformable.

16. A portable system for removing ear wax from an ear canal of a patient, the system comprising:
a speculum having an inner portion nested within an outer portion, the speculum having one or more fluid channels formed between the inner surface of the outer portion and the outer surface of the inner portion, and an inner bore defined at least in part by the inner surface of the inner portion;
a source of fluid in fluid communication with the one or more fluid channels;
a vacuum source in communication with the inner bore for removing fluid and ear wax;
a pump for transferring fluid from the source of fluid to the one or more fluid channels;
an artificial illumination source configured to provide light through the speculum to the ear canal; and
a controller in communication with the pump for delivering fluid to the ear canal according to a predetermined treatment routine.

17. The system of claim 16, further comprising a tool configured to pass through the inner bore of the speculum.

18. The system of claim 17, wherein the tool comprises an elongate body having a distal end and a wire loop extending away from the elongate body.

19. The system of claim 17, wherein the elongate body is hollow and in communication with a vacuum source, the elongate body having a suction port located at the distal end of the elongate body.

20. The system of claim 19, wherein the wire loop is located proximally the suction port.

21. The system of claim 17, wherein the tool comprises a wheel actuator that is configured to extend and retract a portion of the tool by rotation of the wheel actuator.

22. The system of claim 21, wherein the wheel actuator comprises a wheel with a circumferential groove and an elongate and elastic member wrapped around the circumferential groove.

23. The system of claim 16, wherein the artificial illumination source is one or more light emitting diodes.

24. The system of claim 16, further comprising a handle adapter, wherein the handle adapter is reversibly connected to the speculum and provides a gripping surface.

25. The system of claim 24, wherein the handle adapter has a viewing window aligned with the inner bore of the speculum.

26. The system of claim 24, wherein the controller and artificial illumination source are located on the handle adapter.

27. The system of claim 16, wherein the speculum comprises a suction port located on the bottom portion of the speculum, the suction port configured to provide communication between the inner bore and the vacuum source.

28. The system of claim 16, further comprising a compact housing that holds the source of fluid, the vacuum source, and the pump.

29. The system of claim 16, wherein the compact housing is between about 6 to 18 inches in length, 6 to 18 inches in width, and 4 to 12 inches in height.

30. The system of claim 16, further comprising a heating element configured to heat the fluid to a predetermined temperature.

31. A method of removing ear wax from an ear canal of a patient, the method comprising:
inserting a speculum having a longitudinal axis into the ear canal;
illuminating the ear wax within the ear canal with an artificial light source;
viewing the illuminated ear wax through the speculum;
irrigating the interior of the ear canal with one or more jets of irrigation fluid, wherein the one or more jets of irrigation fluid are ejected towards the ear canal walls from the speculum at a predetermined angle away from the longitudinal axis;
dislodging at least a portion of the ear wax with the irrigation fluid while viewing the illuminated ear wax; and
suctioning the irrigation fluid and the dislodged portion of the ear wax from the ear canal.

32. The method of claim 31, further comprising inserting a tool through the speculum to dislodge at least a portion of the ear wax.

33. The method of claim 32, further comprising suctioning at least a portion of the ear wax with the tool.

34. The method of claim 31, wherein the predetermined angle is between about 5 and 45 degrees.

35. The method of claim 31, wherein the ear canal is irrigated with about 300 to 700 mL/min of irrigation fluid.

36. The method of claim 31, wherein the irrigation fluid is delivered at less than 5 psig.

37. The method of claim 31, wherein the irrigation fluid is heated to about 37 degrees Celsius before being used to irrigate the ear canal.

38. The method of claim 31, further comprising actively drying the ear canal after irrigation.

39. The method of claim 31, wherein the step of drying includes introducing a jet of air into the ear canal.

40. The method of claim 31, further comprising creating a negative pressure within the interior of the speculum.
